(19) United States
(12) Patent Application Publication
(10) Pub. No.: US 2007/0249639 A1
(43) Pub. Date: Oct. 25, 2007

(54) Use of 9H-Purine-2,6-Diamine Derivatives in the Treatment of Proliferative Diseases and Novel 9H-Purine-2,6-Diamine Derivatives

(76) Inventors: Rolf Baenteli, Basel (CH); Patrick Chene, Mulhouse (FR); Stephen Paul Collingwood, West Sussex (GB); Pascal Furet, Thann (FR); Peter Meier, Allschwil (CH); Joseph Schoepfer, Riehen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/594,412

(22) PCT Filed: Apr. 4, 2005
(86) PCT No.: PCT/EP05/03521

§ 371 Date: May 30, 2007

(30) Foreign Application Priority Data
Apr. 5, 2004 (GB) 0407723.6

(51) Int. Cl. A61K 31/52; C07D 473/00

(52) U.S. Cl. 514/263.4; 544/264

(57) ABSTRACT

The invention relates to the use of 9H-purine-2,6-diamine compounds and salts thereof in the treatment of proliferative diseases and for the manufacture of pharmaceutical preparations for the treatment of said diseases, pharmaceutical preparations comprising 9H-purine-2,6-diamine compounds, novel 9H-purine-2,6-diamine compounds, and a process for the preparation of the novel 9H-purine-2,6-diamine compounds.

### SUMMARY OF THE INVENTION

The invention relates to methods of use of 9H-purine-2,6-diamine derivatives in the treatment of proliferative diseases, pharmaceutical preparations comprising 9H-purine-2,6-diamine derivatives for the treatment of said diseases, or for the manufacture of pharmaceutical compositions for use in the treatment of said diseases. The present invention also relates to novel 9H-purine-2,6-diamine derivatives, pharmaceutical preparations comprising these 9H-purine-2,6-diamine derivatives, processes for the manufacture of the novel 9H-purine-2,6-diamine derivatives and pharmaceutical preparations, and novel intermediate compound used in the manufacture of 9H-purine-2,6-diamine derivatives.

### BACKGROUND OF THE INVENTION

DNA topoisomerase II is an enzyme which catalyses topological changes in DNA (Wang, J. C., Cellular roles of topoisomerases: a molecular perspective, Nat. Rev. Mol. Cell Biol. 2002; 3:430-40). It has been found in all cell types and it is essential for cell viability. Its role includes the maintenance of intracellular DNA supercoiling, removing supercoils that build up ahead of and behind transcription and replication complexes, and the decatenation of daughter chromosomes following DNA replication. The topoisomerase II action involves cleavage of both DNA strands, transient formation of protein-DNA covalent bonds stabilizing the DNA break, passage of another segment of double stranded DNA through the enzyme-stabilized break, and re-sealing the break at the end of the catalytic process (Champoux, J. J., DNA topoisomerases: structure, function, and mechanism, Annu. Rev. Biochem. 2001 ;70:369-41 3).

Topoisomerase II, which exists as two isoforms, alpha and beta, is an important target in cancer therapy since its disruption is lethal to proliferating tumor cells.

Most topoisomerase II inhibitors kill the tumor cells because they increase the stability of covalent topoisomerase II-cleaved DNA complexes, which under normal conditions appear only transiently. Increased concentration and/or stability of covalent topoisomerase II-cleaved DNA complexes trigger numerous mutagenic, and cytotoxic, events such as insertions, deletions, and illegitimate recombination. These effects in turn are recognized as DNA damage and trigger apoptosis of proliferating cells. Compounds of this class, called topoisomerase II poisons, are therefore more effectively killing tumors cells, which express high topoisomerase II levels (Burden, D. A., Osheroff, N. Mechanism of action of eukaryotic topoisomerase II and drugs targeted to the enzyme, Biochim. Biophys. Acta. 1998;1400:139-54), compared to normal cells. In cells with low topoisomerase II levels, these molecules are less potent. However, because they also induce DNA damaging effects in normal tissues expressing low topoisomerase II levels, they also damage non-tumor cells and therefore they have a relatively narrow therapeutic window.

Another strategy to inhibit cell proliferation via topoisomerase II is to block its catalytic cycle without inducing an accumulation of DNA breaks. Compounds belonging to this class are called catalytic inhibitors (Andoh, T., Ishida, R., Catalytic inhibitors of DNA topoisomerase II, Biochim. Biophys. Acta. 1998;1400:155-71.). ATP competitive inhibitors can block topoisomerase II activity as exemplified by non-hydrolyzable analogs of ATP (Osheroff, N., Sleton, E. R., Brutlag, D. L., DNA topoisomerase II from Drosophyla Melanogaster Relaxation of supercoiled DNA, J. Biol. Chem. 1983;258:9536-43). In the presence of adenyl-5′-yl imidodiphosphate (ADPNP) the enzyme still catalyzes the double-stranded DNA passage but it cannot complete the catalytic cycle. Therefore compounds that bind at the ATP binding site of topoisomerase II have an anticancer effect because they block its enzymatic activity. The advantage of such inhibitors over the poisons is that they are less toxic to normal, non proliferating cells because they do not induce accumulation of DNA breaks, but only inhibit the topoisomerase II catalytic cycle. The design of ATP competitive inhibitors of topoisomerase II is therefore a new strategy to broaden the therapeutic window of antitumor compounds working through this well-established cancer target.

We have now found that the 9H-purine-2,6-diamine residue can be also be used as template for the design of compounds which act as α or β topoisomerase II inhibitors.

There is an ever-existing need to provide novel classes of compounds that can inhibit topoisomerase II and therefore trigger apoptosis of proliferating cells.

### GENERAL DESCRIPTION OF THE INVENTION

The class of 9H-purine-2,6-diamine compounds described herein, especially novel compounds falling under this class, has surprisingly been found to have pharmaceutically advantageous properties, inter alia, as ATP competitive inhibitors or as inhibitors of α or β topoisomerase II.

### DESCRIPTION OF THE FIGURES

FIG. 1: Shows the effect of the compound of Example 1 on DNA relaxation catalyzed by topoisomerase II. The plasmid pUC18 was incubated in the presence of topoisomerase II in the presence or in the absence of 20 μM Example 1. At the indicated times the reaction was stopped and the topology of the DNA analyzed by chromatography on an agarose gel.

### DETAILED DESCRIPTION OF THE INVENTION

The invention in particular relates to 9H-purine-2,6-diamine compounds of the Formula (I):

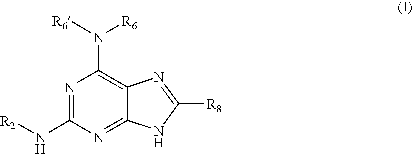

wherein:

R₂ is substituted or unsubstituted lower alkyl, substituted or unsubstituted aryl, substituted or unsubstituted bicyclic aryl, substituted or unsubstituted heteroaryl, substituted or unsubstituted bicyclic heteroaryl;

R′₆ is H or lower alkyl;

R₈ is H, halo, lower alkyl, lower alkenyl, small cycloalkyl, acetyl, —NR₁₂R₁₃ where R₁₂ and R₁₃ are independently H or lower alkyl;

R₆ is substituted or unsubstituted aryl, substituted or unsubstituted heteroaryl, substituted or unsubstituted bicyclic aryl, substituted or unsubstituted bicyclic heteroaryl, or a substituted or unsubstituted aliphatic residue; or R₆ and R′₆ with the N atom form a substituted or unsubstituted heterocyclic radical;

or pharmaceutically acceptable salts thereof,

in the treatment of proliferative diseases, especially those dependent on topoisomerase II activity, or for the manufacture of pharmaceutical compositions for use in the treatment of said diseases, methods of use of compounds of Formula (I) in the treatment of said diseases, pharmaceutical preparations comprising compounds of Formula (I) for the treatment of said diseases, compounds of Formula (I) for use in the treatment of said diseases.

The general terms used hereinbefore and hereinafter preferably have within the context of this disclosure the following meanings, unless otherwise indicated:

“Aryl” is a monocyclic or bicyclic aromatic radical having 6 to 14 carbon atoms, which is unsubstituted or substituted by one or more, preferably one or two substituents, wherein the substituents are as described below. Preferred “aryl” is phenyl and preferred “bicyclic aryl” is napthyl; each of which can be substituted with lower alkyl (such as methyl); lower alkoxy (such as methoxy) and hydroxy.

A “heteroaryl” group is mono-, bi- or tri-cyclic, and comprises 3-24, preferably 4-16 ring atoms, wherein at least one or more, preferably one to four ring carbons are replaced by a heteroatom selected from O, N or S such as oxiranyl, azirinyl, 1,2-oxathiolanyl, imidazolyl, thienyl, furyl, tetrahydrofuryl, indolyl, azetidinyl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, 3H-indolyl, indolyl, benzimidazolyl, benzothiazolyl and benzo[1,2,5]thiadiazolyl, thiacumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being unsubstituted or substituted by one to two radicals selected from the list described below.

Preferably the “monocyclic heteroaryl” group is selected from thiazolyl, pyrazinyl or pyridyl. Preferably the “bicyclic heteroaryl” group is selected from benzothiazolyl, benzo[1,2,5]thiadiazolyl, chromenonyl and quinolyl.

Preferred substituents for the mono- or bi-cyclic heteroaryl include lower alkyl (such as methyl); lower alkyl sulfanyl (such as methaylsulfanyl); and carbonyl.

“Aliphatic” as used herein refers to any non-aromatic carbon based residue. Examples of aliphatic residues include alkyl, cycloalkyl, bicyclic alkyl, tricyclic alkyl, alkenyl and alkynyl, all of which may be substituted or unsubstituted.

“Alkyl” includes lower alkyl preferably alkyl with up to 10 carbon atoms, preferably from 1 to and including 5, and is linear or branched; preferably, lower alkyl is methyl, ethyl, propyl, such as n-propyl or isopropyl, n-butyl, isobutyl, sec-butyl, tert-butyl, straight or branched pentyl, straight or branched hexyl, straight or branched heptyl, straight or branched nonyl or straight or branched decyl. Preferably alkyl is C₁ to C₄-alkyl especially methyl, ethyl, propyl, 2-methyl propyl and t-butyl. The alkyl group may be unsubstituted or substituted with any of the substituents defined below, preferably halo, hydroxy, lower alkoxy (such as methoxy), phenyl, lower alkyl or substituted lower alkyl (such as diphenyl methyl).

A “cycloalkyl” group means C₃ to C₁₀-cycloalkyl having 3 to 8 ring carbon atoms and may be, for example, cyclopropyl, cyclobutyl, cyclopentyl, cyclohexyl, cycloheptyl or cyclooctyl. Preferably, cycloalkyl is cycloheptyl, cyclooctyl or cycloheptyl. The cycloalkyl group may be unsubstituted or substituted with any of the substituents defined below, preferably halo, hydroxy or C₁-C₄ alkyl such as methyl.

A “small cycloalkyl” group means C₃ to C₆-cycloalkyl having 3 to 5 ring carbon atoms and may be, for example, cyclopropyl, cyclobutyl, cyclopropyl.

A “bicyclic alkyl” group means a C₅-C₁₅ alkane derivative composed of two ring structures such as bicyclo[2.2.1]heptyl. The bicyclic alkyl group may be unsubstituted or substituted with any of the substituents defined below.

A “tricyclic alky” group means an C₅-C₂₀ alkane derivative composed of three ring structures, for example adamantanyl. The tricyclic alkyl group may be unsubstituted or substituted with any of the substituents defined below.

“Alkenyl” and “alkynyl” preferably have up to 7 carbon atoms, preferably from 1 to and including 5, and can be linear or branched. Preferred alkenyls include ethylenyl and 2-propenyl(allyl).

“Heterocycl” radical refers to a heterocyclic ring containing 1-4 nitrogen, oxygen or sulfur atoms (e.g. piperazinyl, lower alkyl-piperazinyl, azetidinyl, pyrrolidinyl, piperidino, morpholinyl, imidazolinyl). Heterocyclyl is preferably a heterocyclic radical that is unsaturated, saturated or partially saturated in the bonding ring; has 3-24, more preferably 4-16 ring atoms, wherein at least in the ring bonding to the radical of the molecule of Formula (I) one or more, preferably 1-4, especially one or two carbon ring atoms are replaced by a heteroatom selected from the group consisting of nitrogen, oxygen and sulfur, the bonding ring preferably having 4-12, especially 4-7 ring atoms; heteroaryl being unsubstituted or substituted by one or more, especially 1-4 substituents independently selected from the group consisting of the substituents defined above under “substituted”; especially being a heteroaryl radical selected from the group consisting of indoly, benzofuranyl, thienyl, pyridyl, imidazolinyl, morpholinyl, piperazinyl, piperidino, piperidyl, pyrrolidinyl and azetidinyl, with piperazinyl being especially preferred.

Any of the above defined aryl, bicyclic aryl, heteroaryl, bicyclic heteroaryl, aliphatic, alkyl, cycloalkyl, bicyclic alkyl, tricyclic alkyl, alkenyl, alkynyl or heterocyclic groups may be unsubstituted or independently substituted by up to four, preferably one, two or three substituents, selected from the group consisting of: halo (such as Cl or Br); hydroxy; lower alkyl (such as C₁-C₃ lower alkyl); lower alkyl which may be substituted with any of the substituents defined herein; lower alkenyl; lower alkynyl; lower alkanoyl; alkoxy (such as methoxy); aryl (such as phenyl or benzyl); substituted aryl (such as fluoro phenyl or methoxy phenyl); amino; mono- or disubstituted amino; amino lower alkyl (such as dimethylamino); acetyl amino; amino lower alkoxy (such as ethoxyamine); nitro; cyano; cyano lower alkyl; carboxy; esterified carboxy (such as lower alkoxy carbonyl e.g. methoxy carbonyl); n-propoxy carbonyl or iso-propoxy carbonyl; alkanoyl; benzoyl; carbamoyl; N-mono- or N,N-disubstituted carbamoyl; carbamates; alkyl carbamic acid esters; amidino; guanidine; urea; ureido; mercapto; sulfo; lower alkylthio; sulfoamino; sulfonamide; benzosulfonamide; sulfonate; sulfanyl lower alkyl (such as methyl sulfanyl); sulfoamino; substituted or unsubstituted sulfonamide (such as benzo sulfonamide); substituted or unsubstituted sulfonate (such as chloro-phenyl sulfonate); lower alkylsulfinyl; phenylsulfinyl; phenyl-lower alkylsulfinyl; alkylphenylsulfinyl; lower alkanesulfonyl; phenylsulfonyl; phenyl-lower alkylsulfonyl; alkylphenylsulfonyl; halogen-lower alkylmercapto; halogen-lower alkylsulfonyl; such as especially trifluoromethane sulfonyl; phosphono (—P(═O)(OH)₂); hydroxy-lower alkoxy phosphoryl or di-lower alkoxyphosphoryl; substituted urea (such as 3-trifluoro-methyl-phenyl urea); alkyl carbamic acid ester or carbamates (such as ethyl-N-phenyl-carbamate) or —NR₄R₅, wherein R₄ and R₅ can be the same or different and are independently H; lower alkyl (e.g. methyl, ethyl or propyl); or R₄ and R₅ together with the N atom form a 3- to 8-membered heterocyclic ring containing 1-4 nitrogen, oxygen or sulfur atoms (e.g. piperazinyl, pyrazinyl, lower alkyl-piperazinyl, pyridyl, indolyl, thiophenyl, thiazolyl, n-methyl piperazinyl, benzothiophenyl, pyrrolidinyl, piperidino or imidazolinyl) where the heterocyclic ring may be substituted with any of the substituents defined herein.

Preferred substituents for the above groups include methyl, t-butyl, methoxy, thiazolyl, methaylsulfanyl, carbonyl, hydroxy, phenyl, substituted phenyl, fluorophenyl, pyridyl and pyrazinyl.

Where the plural form is used for compounds, salts, pharmaceutical preparations, diseases and the like, this is intended to mean also a single compound, salt, or the like.

Salts are especially the pharmaceutically acceptable salts of compounds of Formula (I).

Such salts are formed, for example, as acid addition salts, preferably with organic or inorganic acids, from compounds of Formula (I) with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, trifluoroacetic acid, propionic acid, octanoic acid, decanoic acid, dodecanoic acid, glycolic acid, lactic acid, fumaric acid, succinic acid, adipic acid, pimelic acid, suberic acid, azelaic acid, malic acid, tartaric acid, citric acid, amino acids, such as glutamic acid or aspartic acid, maleic acid, hydroxymaleic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalene-disulfonic acid, 2-, 3- or 4-methylbenzenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of Formula (I) may also form internal salts.

For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred.

In view of the close relationship between the compounds in free form and those in the form of their salts, including those salts that can be used as intermediates, for example in the purification or identification of the compounds, tautomers or tautomeric mixtures and their salts, any reference to the compounds hereinbefore and hereinafter especially the compounds of the Formula (I), is to be understood as referring also to the corresponding tautomers of these compounds, especially of compounds of the Formula (I), tautomeric mixtures of these compounds, especially of compounds of the Formula (I), or salts of any of these, as appropriate and expedient and if not mentioned otherwise.

Where “a compound . . . , a tautomer thereof; or a salt thereof” or the like is mentioned, this means “a compound . . . , a tautomer thereof, or a salt of the compound or the tautomer”.

Any asymmetric carbon atom may be present in the (R)—, (S)— or (R,S)-configuration, preferably in the (R)— or (S)-configuration. Substituents at a ring at atoms with saturated bonds may, if possible, be present in cis-(=Z-) or trans (=E-) form. The compounds may thus be present as mixtures of isomers or preferably as pure isomers, preferably as enantiomer-pure diastereomers or pure enantiomers.

### Preferred Embodiments According to the Invention

In the following preferred embodiments, general expression can be replaced by the corresponding more specific definitions provided above and below, thus yielding stronger preferred embodiments of the invention.

Preferred is the USE of compounds of the Formula (I) or pharmaceutically acceptable salts thereof, where the disease to be treated is a proliferative disease depending on topoisomerase II.

The invention relates especially to a compound of the Formula (I):

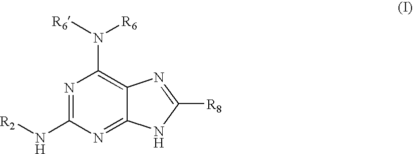

wherein:

R₂ is substituted or unsubstituted lower alkyl, substituted or unsubstituted aryl, substituted or unsubstituted bicyclic aryl, substituted or unsubstituted heteroaryl, substituted or unsubstituted bicyclic heteroaryl;

R′₆ is H or lower alkyl;

R₈ is H, halo, lower alkyl, lower alkenyl, small cycloalkyl, acetyl, —NR₁₂R₁₃ where R₁₂ and R₁₃ are independently H or lower alkyl;

R₆ is substituted or unsubstituted aryl, substituted or unsubstituted heteroaryl, substituted or unsubstituted bicyclic aryl, substituted or unsubstituted bicyclic heteroaryl, or a substituted or unsubstituted aliphatic residue; or R₆ and R′₆ with the N atom form a substituted or unsubstituted heterocyclic radical;

or pharmaceutically acceptable salts thereof,

and use of compounds of Formula (I) in the treatment of proliferative diseases or for the manufacture of pharmaceutical preparations for the treatment of proliferative diseases.

In one embodiment of the present invention, R₂ is aryl or heteroaryl substituted with R′₂ where R′₂ is H or a solubilizing group of the Formula:
—X—Y-A

where X is O, S, —(CH₂)ₙ—, NH or N(lower alkyl);

Y is —(CH₂)ₙ—;

n is 1-4, preferably 2-3; and

A is NR₁₀R₁₁ where R₁₀ and R₁₁ are independently H or C₁-C₃ lower alky, such as methyl, ethyl or propyl, or R₁₀ and R₁₁ with the nitrogen atom form a 3- to 8-membered heterocyclic ring containing 1-4 nitrogen, oxygen or sulfur atoms (e.g. morpholinyl, piperazinyl or lower alkyl-piperazinyl).

In one preferred embodiment A is

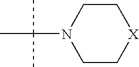

where X is as defined above.

In a further embodiment, R₂ is benzothiazolyl or napthylene substituted with R′₂ as defined above. Examples of R₂ of this embodiment include:

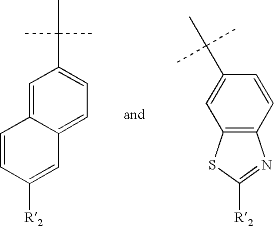

In another embodiment, the invention further relates to a compound of Formula (I) and its use in the treatment of proliferative diseases or for the manufacture of pharmaceutical preparations, wherein:

R₂ is substituted or unsubstituted aryl, substituted or unsubstituted bicyclic aryl, substituted or unsubstituted heteroaryl, substituted or unsubstituted bicyclic heteroaryl;

R′₆ is H or lower alkyl;

R₈ is H, halo, lower alkyl, lower alkenyl, small cycloalkyl, acetyl, —NR₁₂R₁₃ where R₁₂ and R₁₃ are independently H or lower alkyl;

R₆ is substituted or unsubstituted aryl, substituted or unsubstituted heteroaryl, substituted or unsubstituted bicyclic aryl, substituted or unsubstituted bicyclic heteroaryl, or a substituted or unsubstituted aliphatic residue; or R₆ and R′₆ with the N atom form a substituted or unsubstituted heterocyclic radical;

or pharmaceutically acceptable salts thereof.

The present invention also relates to a compound of Formula (I) wherein:

R₂ is substituted or unsubstituted aryl, substituted or unsubstituted bicyclic aryl, substituted or unsubstituted heteroaryl, substituted or unsubstituted bicyclic heteroaryl;

R′₆ is H or lower alkyl;

R₈ is H, halo, lower alkyl, lower alkenyl, small cycloalkyl, acetyl, —NR₁₂R₁₃ where R₁₂ and R₁₃ are independently H or lower alkyl;

R₆ is bicyclic alkyl, tricyclic alkyl, or heteroaryl, all of which may be substituted or unsubstituted, preferably substituted with a heteroaryl; or pharmaceutically acceptable salts thereof, and use of compounds of Formula (I) in the treatment of proliferative diseases or for the manufacture of pharmaceutical preparations for the treatment of proliferative diseases.

In a further embodiment, the invention also relates to a compound of the Formula (I), wherein:

R₂ substituted or unsubstituted aryl, substituted or unsubstituted bicyclic aryl, substituted or unsubstituted heteroaryl, substituted or unsubstituted bicyclic heteroaryl;

R′₆ is H or lower alkyl;

R₈ is H, halo, lower alkyl, lower alkenyl, small cycloalkyl, acetyl, —NR₁₂R₁₃ where R₁₂ and R₁₃ are independently H or lower alkyl;

R₆ is substituted or unsubstituted aryl, substituted or unsubstituted heteroaryl, substituted or unsubstituted bicyclic aryl, substituted or unsubstituted bicyclic heteroaryl, or a alkyl, cycloalkyl, bicyclic alkyl, tricyclic alkyl, alkenyl and alkynyl, all of which may be substituted or unsubstituted; or R₆ and R′₆ with the N atom form a substituted or unsubstituted heterocyclic radical;

or pharmaceutically acceptable salts thereof,

and use of compounds of Formula (I) in the treatment of proliferative diseases or for the manufacture of pharmaceutical preparations for the treatment of proliferative diseases or especially for use in the diagnostic or therapeutic treatment of a warm-blooded animal, especially a human.

The invention further relates to a compound of the Formula (I), and uses thereof, wherein

R₂ is phenyl; phenyl substituted with thiazolyl; benzothiazolyl; benzothioazoyl substituted with lower alkyl such as methyl or t-butyl or substituted with lower alkyl sulfanyl such as methyl sulfanyl; quinolinyl; quinolinyl substituted with methyl; naphthyl; indolyl; benzo[1,2,5]thiadiazolyl; chromenyl; chromen-2-one or amino chromen-2-one;

R′₆ is H or lower alkyl;

R₈ is halo, cyclopropyl, cyclopentyl, 2-methyl propyl, methyl, ethyl, t-butyl, ethylenyl, allyl, acetyl, —NHMe or —NH₂;

R₆ is cycloheptyl; cyclooctyl; cycloheptyl; cyclohexyl or cyclohexyl substituted with hydroxy; adamantanyl; bicyclo[2.2.1]heptyl; phenyl or phenyl substituted with lower alkoxy, e.g. methoxy; quinolinyl; lower alkyl such as t-butyl; 2,2,2-trifluoro-1-methyl-ethyl-; methyl or methyl substituted with diphenyl; ethyl or ethyl substituted with methyl and fluorophenyl, e.g. 2-(fluoro-phenyl)-1,1-dimethyl-ethyl; propyl or propyl substituted with methyl or hydroxy e.g. 1,1-dimethyl propyl or 1-hydroxy-2-methyl-prop-2-yl; lower aliphatic ester e.g. 3-yl-butyric acid ethyl ester or amide 3-yl-butyramide;

R₆ R₆N is piperazinyl substituted with pyridine or pyrazine; or pyrrolidin-1-yl e.g. 2-methyl-pyrrolidin-1-yl;

or pharmaceutically acceptable salts thereof.

In another embodiment, the invention relates to a compound of the Formula (I), wherein

R₂ is phenyl; phenyl substituted with thiazolyl; benzothiazolyl; benzothioazoyl substituted with lower alkyl such as methyl or t-butyl or substituted with lower alkyl sulfanyl such as methyl sulfanyl; quinolinyl; quinolinyl substituted with methyl; naphthyl; indolyl; benzo[1,2,5]thiadiazolyl; chromenyl; chromen-2-one or amino chromen-2-one;

R′₆ is H or lower alkyl;

R₈ is lower alkyl or small cycloalkyl;

R₆ is cycloheptyl; cyclooctyl; cycloheptyl; cyclohexyl or cyclohexyl substituted with hydroxy; adamantanyl; bicyclo[2.2.1] heptyl; phenyl or phenyl substituted with lower alkoxy, e.g. methoxy; quinolinyl; lower alkyl such as t-butyl; 2,2,2-trifluoro-1-methyl-ethyl-; methyl or methyl substituted with diphenyl; ethyl or ethyl substituted with methyl and fluorophenyl, e.g. 2-(fluoro-phenyl)-1,1-dimethyl-ethyl; propyl or propyl substituted with methyl or hydroxy e.g. 1,1-dimethyl propyl or 1-hydroxy-2-methyl-prop-2-yl; lower aliphatic ester e.g. 3-yl-butyric acid ethyl ester or amide 3-yl-butyramide;

R₆ R_{′6}N is piperazinyl substituted with pyridine or pyrazine; or pyrrolidin-1-yl e.g. 2-methyl-pyrrolidin-1-yl;

or pharmaceutically acceptable salts thereof, and uses thereof in the treatment of proliferative diseases or for the manufacture of pharmaceutical preparations.

The present invention is also directed to novel intermediates of the Formula (II):

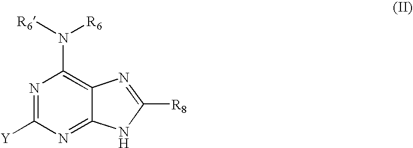

wherein R′₆ and R₆ are as defined above, R₈ is H or lower alkyl, small cycloalkyl, or R₈ is as defined above, and Y is a protecting group selected from chlorine, bromine or iodine, preferably chlorine, with the proviso that if R₈ is H then R₆ cannot be bicyclo[2.2.1]hept-2-ylamine, methoxyphenyl or phenyl. Preferably R₆ is cycloheptyl, cyclooctyl, cyclohexanyl, adamantanyl, 2-methyl-propanol, quinolinyl, t-butyl, 1-hydroxy-methylpropyl, 4-hydroxy-cyclohexyl, C,C-diphenyl methyl, 2,2,2-trifluoro-1-methyl-ethyl, 2-methyl-pyrrolidin-1-yl, 3-yl-butyramide, or 3-yl-butyric acid ethyl ester.

The present invention is also directed to novel intermediates of the Formula (III):

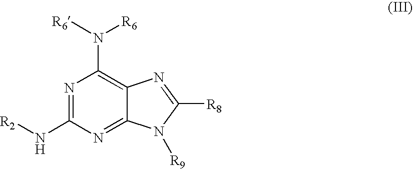

wherein R₂, R₈, R′₆ and R₆ are as defined above and R₉ is a protecting group. Preferably R₂ is quinolinyl, methyl-quinolinyl, benzothiazolyl, methyl benzothiazolyl, t-butyl benzothiazolyl, napthyl, 6-methoxy-naphthalen-2-yl, 6-(2-morpholin-4-yl-ethoxy)-naphthalen-2-yl, 2-methyl-1-BOC-indol-5-yl, 1-BOC-Indol-5-yl, and 2-(2-morpholin-4-yl-ethoxy)-benzothiazol-6-yl; R₈ is H, ethyl, isopropyl, cyclopropyl, cyclopentyl, 2,4-dimethoxy-benzylamino, (2,4-dimethoxy-benzyl)-methyl-amino and 1-ethoxyvinyl; and R′₆ are hydrogen; R₆ is t-butyl or cycloheptyl and R₉ is tetrahydropyranyl or bis-(4-methoxy-phenyl)-methyl.

The present invention is also directed to novel intermediates of the Formula (IV):

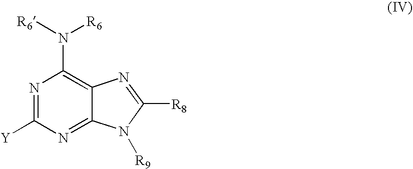

wherein Y, R₈, R₉, R′₆ and R₆ are as defined above. Preferably R₈ is H, ethyl, isopropyl, cyclopropyl, cyclopentyl, 2,4-dimethoxy-benzylamino, (2,4-dimethoxy-benzyl)-methyl-amino and 1-ethoxyvinyl; R₉ is tetrahydropyranyl or bis-(4-methoxy-phenyl)-methyl; R′₆ is hydrogen and R₆ is C,C-diphenylmethyl, t-butyl or cycloheptyl.

The present invention is also directed to novel intermediates of the Formula (V):

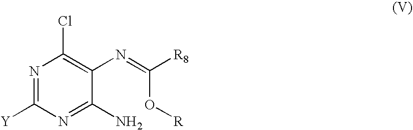

R₈ and Y are as defined above, preferably R₈ is lower alkyl such as methyl or ethyl; or small cycloalky such as cyclopropyl or cyclopentyl. R is lower alkyl, for example methyl or ethyl.

The present invention is also directed to a compound chosen from the group consisting of;

N*2*-Benzothiazol-6-yl-N*6*-cycloheptyl-9H-purine-2,6-diamine;

N*6*-Cycloheptyl-N*2*-(4-thiazol-2-yl-phenyl)-9H-purine-2,6-diamine;

N*6*-Cycloheptyl-N*2*-quinolin-6-yl-9H-purine-2,6-diamine;

2-[2-(Benzothiazol-6-ylamino)-9H-purin-6-ylamino]-2-methyl-propan-1-ol;

N*6*-Adamantan-2-yl-N*2*-benzothiazol-6-yl-9H-purine-2,6-diamine;

N*2*-Benzothiazol-6-yl-N*6*-bicyclo[2.2.1]hept-2-yl-9H-purine-2,6-diamine;

N*2*-Benzothiazol-6-yl-N*6*-cyclooctyl-9H-purine-2,6-diamine;

N*2*-Benzothiazol-6-yl-N*6*-(3-methoxy-phenyl)-9H-purine-2,6-diamine;

4-[2-(Benzothiazol-6-ylamino)-9H-purin-6-ylamino]-cyclohexanol;

N*2*,N*6*-Di-quinolin-6-yl-9H-purine-2,6-diamine;

N*6*-Benzhydryl-N*2*-benzothiazol-6-yl-9H-purine-2,6-diamine;

N*6*-Phenyl-N*2*-quinolin-6-yl-9H-purine-2,6-diamine;

N*6*-Benzhydryl-N*2*-quinolin-6-yl-9H-purine-2,6-diamine;

N*6*-tert-Butyl-N*2*-naphthalen-2-yl-9H-purine-2,6-diamine;

N*6*-tert-Butyl-N*2*-(2-methyl-quinolin-6-yl)-9H-purine-2,6-diamine;

N*6*-tert-Butyl-N*2*-(2-methyl-benzothiazol-5-yl)-9H-purine-2,6-diamine;

N*6*-tert-Butyl-N*2*-(2-tert-butyl-benzothiazol-6-yl)-9H-purine-2,6-diamine;

N*6*-tert-Butyl-N*2*-(2-methyl-benzothiazol-6-yl)-9H-purine-2,6-diamine;

N*2*-(4-Benzothiazol-2-yl-phenyl)-N*6*-tert-butyl-9H-purine-2,6-diamine;

N*6*-tert-Butyl-N*2*-(6-methoxy-naphthalen-2-yl)-9H-purine-2,6-diamine;

N*6*-tert-Butyl-N*2*-[6-(2-morpholin-4-yl-ethoxy)-naphthalen-2-yl]-9H-purine-2,6-diamine;

N*6*-tert-Butyl-N*2*-(2-methyl-1H-indol-5-yl)-9H-purine-2,6-diamine;

N*6*-tert-Butyl-N*2*-(1H-indol-5-yl)-9H-purine-2,6-diamine;

N*2*-Benzothiazol-6-yl-N*6*-tert-butyl-8-methyl-9H-purine-2,6-diamine;

N*2*-Benzothiazol-6-yl-N*6*-cycloheptyl-8-methyl-9H-purine-2,6-diamine;

N*2*-Benzothiazol-6-yl-N*6*-tert-butyl-8-ethyl-9H-purine-2,6-diamine;

N*2*-Benzothiazol-6-yl-8-ethyl-N*6*-(2,2,2-trifluoro-1-methyl-ethyl)-9H-purine-2,6-diamine;

Benzothiazol-6-yl-[8-ethyl-6-(2-methyl-pyrrolidin-1-yl)-9H-purin-2-yl]-amine;

3-[2-(Benzothiazol-6-ylamino)-8-ethyl-9H-purin-6-ylamino]-butyramide;

3-[2-(Benzothiazol-6-ylamino)-8-ethyl-9H-purin-6-ylamino]-butyric acid ethyl ester;

N*2*-Benzothiazol-6-yl-N*6*-tert-butyl-8-propyl-9H-purine-2,6-diamine;

N*2*-Benzothiazol-6-yl-N*6*-tert-butyl-8-isopropyl-9H-purine-2,6-diamine;

N*2*-Benzothiazol-6-yl-N*6*-tert-butyl-8-cyclopentyl-9H-purine-2,6-diamine;

6-(6-tert-Butylamino-8-ethyl-9H-purin-2-ylamino)-chromen-2-one;

N*6*-tert-Butyl-8-ethyl-N*2*-(2-methylsulfanyl-benzothiazol-6-yl)-9H-purine-2,6-diamine;

N*6*-tert-Butyl-8-ethyl-N*2*-[2-(2-morpholin-4-yl-ethoxy)-benzothiazol-6-yl]-9H-purine-2,6-diamine;

N*6*-tert-Butyl-8-isopropyl-N*2*-{1-methylene-3-[5-methyl-2-(2-morpholin-4-yl-ethoxy)-thiazol-4-yl]-allyl}-9H-purine-2,6-diamine;

N*6*-tert-Butyl-8-cyclopropyl-N*2*-{1-methylene-3-[5-methyl-2-(2-morpholin-4-yl-ethoxy)-thiazol-4-yl]-allyl}-9H-purine-2,6-diamine;

N*6*-tert-Butyl-8-cyclopentyl-N*2*-{1-methylene-3-[5-methyl-2-(2-morpholin-4-yl-ethoxy)-thiazol-4-yl]-allyl}-9H-purine-2,6-diamine;

N*2*-Benzothiazol-6-yl-N*6*-tert-butyl-8-cyclopropyl-9H-purine-2,6-diamine;

N*2*-Benzothiazol-6-yl-N*6*-tert-butyl-9H-purine-2,6,8-triamine;

N*6*-tert-Butyl-N*2*-naphthalen-2-yl-9H-purine-2,6,8-triamine;

N*6*-tert-Butyl-N*8*-methyl-N*2*-naphthalen-2-yl-9H-purine-2,6,8-triamine;

1-[2-(Benzothiazol-6-ylamino)-6-tert-butylamino-9H-purin-8-yl]-ethanone;

N*6*-tert-Butyl-N*2*-quinolin-6-yl-9H-purine-2,6-diamine;

N*2*-Benzo[1,2,5]thiadiazol-5-yl-N*6*-tert-butyl-9H-purine-2,6-diamine;

N*6*-tert-Butyl-N*2*-(2-methyl-benzothiazol-6-yl)-9H-purine-2,6-diamine;

N*2*-Benzo[1,2,5]thiadiazol-5-yl-N*6*-cycloheptyl-9H-purine-2,6-diamine;

N*6*-Cycloheptyl-N*2*-(2-methyl-benzothiazol-6-yl)-9H-purine-2,6-diamine;

N*2*-Benzothiazol-6-yl-N*6*-(1,1-dimethyl-propyl)-9H-purine-2,6-diamine;

N*6*-(1,1-Dimethyl-propyl)-N*2*-quinolin-6-yl-9H-purine-2,6-diamine;

N*2*-Benzo[1,2,5]thiadiazol-5-yl-N*6*-(1,1-dimethyl-propyl)-9H-purine-2,6-diamine;

N*6*-(1,1-Dimethyl-propyl)-N*2*-(2-methyl-benzothiazol-6-yl)-9H-purine-2,6-diamine;

N*2*-Benzothiazol-6-yl-N*6*-[2-(4-fluoro-phenyl)-1,1-dimethyl-ethyl]-9H-purine-2,6-diamine;

N*6*-[2-(4-Fluoro-phenyl)-1,1-dimethyl-ethyl]-N*2*-quinolin-6-yl-9H-purine-2,6-diamine;

N*2*-Benzo[1,2,5]thiadiazol-5-yl-N*6*-[2-(4-fluoro-phenyl)-1,1-dimethyl-ethyl]-9H-purine-2,6-diamine;

N*6*-[2-(4-Fluoro-phenyl)-1,1-dimethyl-ethyl]-N*2*-(2-methyl-benzothiazol-6-yl)-9H-purine-2,6-diamine;

Benzothiazol-6-yl-[6-(4-pyridin-3-yl-piperazin-1-yl)-9H-purin-2-yl]-amine;

Benzothiazol-6-yl-[6-(4-pyridin-2-yl-piperazin-1-yl)-9H-purin-2-yl]-amine;

[6-(4-Pyridin-2-yl-piperazin-1-yl)-9H-purin-2-yl]-quinolin-6-yl-amine;

Benzo[1,2,5]thiadiazol-5-yl-[6-(4-pyridin-2-yl-piperazin-1-yl)-9H-purin-2-yl]-amine;

(2-Methyl-benzothiazol-6-yl)-[6-(4-pyridin-2-yl-piperazin-1-yl)-9H-purin-2-yl]-amine;

Quinolin-6-yl-[6-(2,3,5,6-tetrahydro-[1,2′]bipyrazinyl-4-yl)-9H-purin-2-yl]-amine;

N*2*-Benzothiazol-6-yl-N*6*-tert-butyl-9H-purine-2,6-diamine;

N*2*-Benzothiazol-6-yl-N*6*-cycloheptyl-9H-purine-2,6-diamine;

N*6*-Cycloheptyl-N*2*-quinolin-6-yl-9H-purine-2,6-diamine;

2-[2-(Benzothiazol-6-ylamino)-9H-purin-6-ylamino]-2-methyl-propan-1-ol;

8-Bromo-N*6*-tert-butyl-N*2*-naphthalen-2-yl-9H-purine-2,6-diamine;

N*6*-tert-Butyl-N*2*-naphthalen-2-yl-8-vinyl-9H-purine-2,6-diamine;

8-Allyl-N*6*-tert-butyl-N*2*-naphthalen-2-yl-9H-purine-2,6-diamine;

N*6*-tert-Butyl-8-methyl-N*2*-naphthalen-2-yl-9H-purine-2,6-diamine;

N*6*-tert-Butyl-8-ethyl-N*2*-naphthalen-2-yl-9H-purine-2,6-diamine;

N*6*-tert-Butyl-8-isobutyl-N*2*-naphthalen-2-yl-9H-purine-2,6-diamine;

and pharmaceutically acceptable salts thereof.

Furthermore, the invention relates to a compound chosen from the group consisting of:

(2-Chloro-9H-purin-6-yl)-cycloheptyl-amine;

2-(2-Chloro-9H-purin-6-ylamino)-2-methyl-propan-1-ol;

Adamantan-2-yl-(2-chloro-9H-purin-6-yl)-amine;

(2-Chloro-9H-purin-6-yl)-cyclooctyl-amine;

4-(2-Chloro-9H-purin-6-ylamino)-cyclohexanol;

(2-Chloro-9H-purin-6-yl)-quinolin-6-yl-amine;

Benzhydryl-(2-chloro-9H-purin-6-yl)-amine;

N*6*-Benzhydryl-N*2*-quinolin-6-yl-9-(tetrahydro-pyran-2-yl)-9H-purine-2,6-diamine;

N*2*-Benzothiazol-6-yl-N*6*-tert-butyl-9-(tetrahydro-pyran-2-yl)-9H-purine-2,6-diamine;

N*6*-tert-Butyl-N*2*-naphthalen-2-yl-9-(tetrahydro-pyran-2-yl)-9H-purine-2,6-diamine;

N*6*-tert-Butyl-N*2*-(2-methyl-quinolin-6-yl)-9-(tetrahydro-pyran-2-yl)-9H-purine-2,6-diamine;

N*6*-tert-Butyl-N*2*(2-methyl-benzothiazol-5-yl)-9-(tetrahydro-pyran-2-yl)-9H-purine-2,6-diamine;

N*6*-tert-Butyl-N*2*-(2-tert-butyl-benzothiazol-6-yl)-9-(tetrahydro-pyran-2-yl)-9H-purine-2,6-diamine;

N*6*-Cycloheptyl-N*2*-quinolin-6-yl-9-(tetrahydro-pyran-2-yl)-9H-purine-2,6-diamine;

N*2*-(4-Benzothiazol-2-yl-phenyl)-N*6*-tert-butyl-9-(tetrahydro-pyran-2-yl)-9H-purine-2,6-diamine;

N*6*-tert-Butyl-N*2*-(6-methoxy-naphthalen-2-yl)-9-(tetrahydro-pyran-2-yl)-9H-purine-2,6-diamine;

N*6*-tert-Butyl-N*2*-[6-(2-morpholin-4-yl-ethoxy)-naphthalen-2-yl]-9-(tetrahydro-pyran-2)-9H-purine-2,6-diamine;

5-{9-[Bis-(4-methoxy-phenyl)-methyl]-6-tert-butylamino-9H-purin-2-ylamino}-2-methyl-indole-1-carboxylic acid tert-butyl ester;

9-[Bis-(4-methoxy-phenyl)-methyl]-N*6*-tert-butyl-N*2*-(1H-indol-5-yl)-9H-purine-2,6-diamine;

9-[Bis-(4-methoxy-phenyl)-methyl]-N*6*-tert-butyl-8-ethyl-N*2*-[2-(2-morpholin-4-yl-ethoxy)-benzothiazol-6-yl]-9H-purine-2,6-diamine;

N*6*-tert-Butyl-8-ethyl-N*2*-[2-(2-morpholin-4-yl-ethoxy)-benzothiazol-6-yl]-9-(tetrahydro-pyran-2-yl)-9H-purine-2,6-diamine;

N*6*-tert-Butyl-8-isopropyl-N*2*-{1-methylene-3-[5-methyl-2-(2-morpholin-4-yl-ethoxy)-thiazol-4-yl]-allyl}-9-(tetrahydro-pyran-2-yl)-9H-purine-2,6-diamine;

N*6*-tert-Butyl-8-cyclopropyl-N*2*-{1-methylene-3-[5-methyl-2-(2-morpholin-4-yl-ethoxy)-thiazol-4-yl]-allyl}-9-(tetrahydro-pyran-2-yl)-9H-purine-2,6-diamine;

N*6*-tert-Butyl-8-cyclopentyl-N*2*-{1-methylene-3-[5-methyl-2-(2-morpholin-4-yl-ethoxy)-thiazol-4-yl]-allyl}-9-(tetrahydro-pyran-2-yl)-9H-purine-2,6-diamine;

N*2*-Benzothiazol-6-yl-N*6*-tert-butyl-8-cyclopropyl-9-(tetrahydro-pyran-2-yl)-9H-purine-2,6-diamine;

N*2*-Benzothiazol-6-yl-N*6*-tert-butyl-N*8*-(2,4-dimethoxy-benzyl)-9-(tetrahydro-pyran-2-yl)-9H-purine-2,6,8-triamine;

N*6*-tert-Butyl-N*8*-(2,4-dimethoxy-benzyl)-N*2*-naphthalen-2-yl-9-(tetrahydro-pyran-2-yl)-9H-purine-2,6,8-triamine;

N*6*-tert-Butyl-N*8*-(2,4-dimethoxy-benzyl)-N*8*-methyl-N*2*-naphthalen-2-yl-9-(tetrahydro-pyran-2-yl)-9H-purine-2,6,8-triamine;

N*2*-Benzothiazol-6-yl-N*6*-tert-butyl-8-(1-ethoxy-vinyl)-9-(tetrahydro-pyran-2-yl)-9H-purine-2,6-diamine;

tert-Butyl-(2-chloro-8-methyl-9H-purin-6-yl)-amine;

(2-Chloro-8-methyl-9H-purin-6-yl)-cycloheptyl-amine;

tert-Butyl-(2-chloro-8-ethyl-9H-purin-6-yl)-amine;

(2-Chloro-8-ethyl-9H-purin-6-yl)-(2,2,2-trifluoro-1-methyl-ethyl)-amine;

2-Chloro-8-ethyl-6-(2-methyl-pyrrolidin-1-yl)-9H-purine;

3-(2-Chloro-8-ethyl-9H-purin-6-ylamino)-butyramide;

3-(2-Chloro-8-ethyl-9H-purin-6-ylamino)-butyric acid ethyl ester;

tert-Butyl-(2-chloro-8-propyl-9H-purin-6-yl)-amine;

tert-Butyl-(2-chloro-8-isopropyl-9H-purin-6-yl)-amine;

tert-Butyl-(2-chloro-8-cyclopentyl-9H-purin-6-yl)-amine;

tert-Butyl-(2-chloro-8-cyclopropyl-9H-purin-6-yl)-amine;

Benzhydryl-[2-chloro-9-(tetrahydro-pyran-2-yl)-9H-purin-6-yl]-amine;

tert-Butyl-[2-chloro-9-(tetrahydro-pyran-2-yl)-9H-purin-6-yl]-amine;

[2-Chloro-9-(tetrahydro-pyran-2-yl)-9H-purin-6-yl]-cycloheptyl-amine;

{9-[Bis-(4-methoxy-phenyl)-methyl]-2-chloro-9H-purin-6-yl)-tert-butyl-amine;

{9-[Bis-(4-methoxy-phenyl)-methyl]-2-chloro-8-ethyl-9H-purin-6-yl}-tert-butyl-amine;

tert-Butyl-[2-chloro-8-ethyl-9-(tetrahydro-pyran-2-yl)-9H-purin-6-yl]-amine;

tert-Butyl-[2-chloro-8-isopropyl-9-(tetrahydro-pyran-2-yl)-9H-purin-6-yl]-amine;

tert-Butyl-[2-chloro-8-cyclopropyl-9-(tetrahydro-pyran-2-yl)-9H-purin-6-yl]-amine;

tert-Butyl-[2-chloro-8-cyclopentyl-9-(tetrahydro-pyran-2-yl)-9H-purin-6-yl]-amine;

tert-Butyl-[2-chloro-8-cyclopropyl-9-(tetrahydro-pyran-2-yl)-9H-purin-6-yl]-amine;

N*6*-tert-Butyl-2-chloro-N*8*-(2,4-dimethoxy-benzyl)-9-(tetrahydro-pyran-2-yl)-9H-purine-6,8-diamine;

N*6*-tert-Butyl-2-chloro-N*8*-(2,4-dimethoxy-benzyl)-N*8*-methyl-9-(tetrahydro-pyran-2-yl)-9H-purine-6,8-diamine;

tert-Butyl-[2-chloro-8-(1-ethoxy-vinyl)-9-(tetrahydro-pyran-2-yl)-9H-purin-6-yl]-amine;

9-[Bis-(4-methoxy-phenyl)-methyl]-2,6-dichloro-9H-purine;

N-(4-Amino-2,6-dichloro-pyrimidin-5-yl)-acetimidic acid ethyl ester;

N-(4-Amino-2,6-dichloro-pyrimidin-5-yl)-propionimidic acid ethyl ester;

N-(4-Amino-2,6-dichloro-pyrimidin-5-yl)-butyrimidic acid methyl ester;

N-(4-Amino-2,6-dichloro-pyrimidin-5-yl)-2-methyl-propionimidic acid ethyl ester;

N-(4-Amino-2,6-dichloro-pyrimidin-5-yl)-cyclopentanecarboximidic acid ethyl ester;

N-(4-Amino-2,6-dichloro-pyrimidin-5-yl)-cyclopropanecarboximidic acid ethyl ester;

6-(2-Morpholin-4-yl-ethoxy)-naphthalen-2-ylamine;

4-[2-(6-Bromo-naphthalen-2-yloxy)-ethyl]-morpholine;

5-Amino-2-methyl-indole-1-carboxylic acid tert-butyl ester;

2-Methyl-5-nitro-indole-1-carboxylic acid tert-butyl ester;

2-(2-Morpholin-4-yl-ethoxy)-benzothiazol-6-ylamine;

2-(2-Morpholin-4-yl-ethoxy)-6-nitro-benzothiazole;

[8-Bromo-2-chloro-9-(tetrahydro-pyran-2-yl)-9H-purin-6-yl]-tert-butyl-amine;

8-Bromo-2,6-dichloro-9-(tetrahydro-pyran-2-yl)-9H-purine; and

2,6-Dichloro-8-(1-ethoxy-vinyl)-9-(tetrahydro-pyran-2-yl)-9H-purine.

The present invention is also directed to use of the above intermediates in a process to prepare a compound of Formula (I).

Where subsequently the term “USE” is mentioned, this includes any one or more of the following embodiments of the invention, respectively: the use in the treatment of proliferative diseases, especially those dependant on topoisomerase II activity, the use for the manufacture of pharmaceutical compositions for use in the treatment of said diseases, pharmaceutical preparations comprising 9H-purine-2,6-diamine derivatives for the treatment of said diseases, and 9H-purine-2,6-diamine derivatives for use in the treatment of said diseases, as appropriate and expedient, if not stated otherwise. In particular, diseases to be treated and are thus preferred for USE of a compound of Formula (I) are selected from proliferative diseases, more especially diseases that depend on topoisomerase II activity.

In a broader sense of the invention, a proliferative disease includes hyperproliferative conditions, such as leukemias, hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. Proliferative diseases also include tumors with low levels of topoisomerase II activity.

Very preferred is a method of treating a proliferative disease, preferably a benign or especially malignant tumor, more preferably carcinoma of the brain, kidney, liver, adrenal gland, bladder, breast, stomach (especially gastric tumors), ovaries, colon, rectum, prostate, pancreas, lung (especially SCLC), vagina, thyroid, sarcoma, glioblastomas, multiple myeloma or gastrointestinal cancer, especially colon carcinoma or colorectal adenoma, or a tumor of the neck and head, an epidermal hyperproliferation, especially psoriasis, prostate hyperplasia, a neoplasia, especially of epithelial character, preferably mammary carcinoma, or a leukemia. Most preferred are breast tumors with over-expressed ErbB-2 and low topoisomerase II levels.

Compounds of Formula (I) are able to bring about the regression of tumors and to prevent the formation of tumor metastases and the growth of (also micro)metastases. In addition they can be used in epidermal hyperproliferation (e.g. psoriasis), in prostate hyperplasia, and in the treatment of neoplasias, especially of epithelial character, for example mammary carcinoma.

The compounds of Formula (I) have valuable pharmacological properties and are useful in the treatment of proliferative diseases.

The inhibition of topoisomerase II is measured as follows:

Purified human topoisomerase II α was purchased from Professor Neil Osheroff (Vanderbilt University—Nashville, Tenn., USA).

The pUC18 (Stratagene) plasmid was introduced into Escherichia coil XL-1 (Stratagene) and purified with the HiSpeed Plasmid Purification kit (Qiagen) according to the manufacturer instructions. The purity of the DNA preparations was evaluated spectrophotometrically (OD₂₆₀/OD₂₈₀ ratio) and by agarose gel.

Preparation of the Malachite Green Reagent:

Three volumes of 0.045% malachite green (Sigma, catalog no. M-9636) diluted in water are mixed for 20 min at room temperature with one volume of 4.2% ammonium molybdate (Axon Lab AG, catalog no. A-2246) diluted in 4 N HCl. After mixing, Triton X-100 (Merck, catalog no. 1.12298) is added to a final concentration of 0.01%. The solution is filtered at 0.2 μm and stored at room temperature in the dark.

ATPase Assay:

ATP hydrolysis was monitored by measuring the production of inorganic phosphate released during the catalytic cycle of topoisomerase II α with acidic molybdate and malachite green (Lanzetta,et al., An improved assay for nanomole amounts of inorganic phosphate, Anal Biochem 1979;100:95-7). Briefly, human topoisomerase II α is pre-incubated at 37° C. for 10 min in 90 μl reaction buffer (10 mM Tris.HCl pH 7.9, 175 mM KCl, 1 mM EDTA, 2 mM DTT, 5 mM MgCl₂, 2.5% glycerol) in the presence of the indicated amount of pUC18 and DMSO in F96 maxisorp Nunclmmuno plates (Nunc). The reaction is initiated by the addition of 10 μl ATP at the indicated concentrations and is carried out for 30 min at 37° C. under constant agitation. The reaction is stopped by the addition of 200 μl molybdate/malachite green solution and the absorbance is immediately measured at 630 nm. The OD₆₃₀ value is measured in the absence of protein for each ATP concentration (background) is subtracted from the value obtained in the presence of enzyme. A standard curve with inorganic phosphate is used to determine the amount of inorganic phosphate produced during the reaction and to ensure that the measures are in the linear range of the assay. The kinetic parameters of the enzymes are determined from measurements (at least in duplicate) of the initial rates at different ATP concentrations. The data are analyzed with GraFit (Erithacus Software).

The compounds are tested at 10 μM of concentration to determine their inhibition potential given % of inhibition of the TOPO II ATPase activity.

For the determination of the IC₅₀, the procedure is repeated for different concentrations of test compound selected to cover the range of 0% to 100% inhibition and the concentration at which 50% inhibition of topoisomerase II occurs (IC₅₀) for each compound is determined from concentration-inhibition curves in a conventional manner.

For instance, the compounds of Examples 1, 15, 22 and 25 hereinbelow have IC₅₀ values of 8.4 μM, 3.3 μM, 1.8 μM, and 2.1 μM respectively. Examples 27, 35 and 36 have IC₅₀ values of 0.6 μM, 2.8 μM and 0.9 μM respectively in the ATPase assay.

DNA Relaxation Assay:

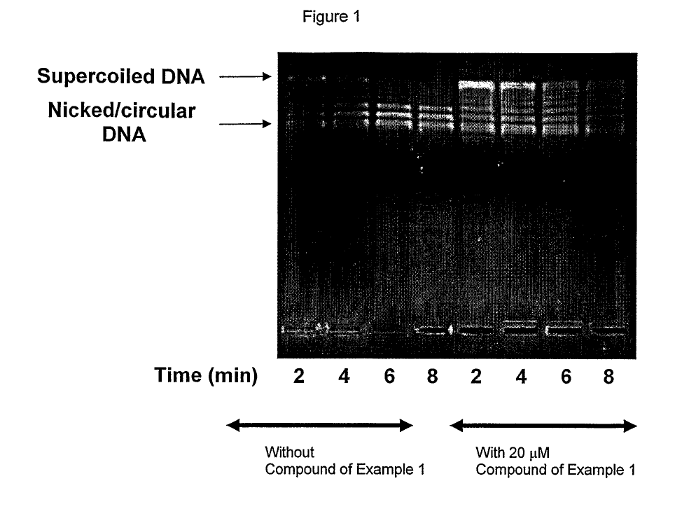

Human topoisomerase II ∝ (45 ng) is pre-incubated at 37° C. for 5 min in 15 μl reaction buffer (10 mM Tris.HCl pH 7.9, 175 mM KCl, 1 mM EDTA, 2 mM DTT, 5 mM MgCl₂, 2.5% glycerol) in the presence of 600 ng pUC18 and 3% DMSO in 0.5 ml eppendorf. The reaction is initiated by the addition of 5 μl of 1.6 mM ATP and carried out at 37° C. At 2, 4, 6 and 8 minutes, the reaction is stopped by the addition of 3.3 μl stop reaction (Blue/Orange loading dye (Promega) containing 100 mM EDTA and 0.5% SDS) and the reaction mixture is loaded onto a 1% agarose gel. The gel is run for 2 h at 5 V/cm and stained for 30 min with an aqueous solution of ethidium bromide (1 μg/ml). The bands are visualized by transillumination with ultraviolet light. See FIG. 1.

Synthetic Procedure

Compounds of Formula (I) are prepared by:

(A) reacting a substituted 9H-purin-6-ylamine of Formula (II) with an hereroaryl/aryl-amine to form a compound of Formula (I), where R′₆, R₆, R₂ and R₈ are as defined above, and Y is a leaving group, preferably halogen such as bromine, iodine or, in particular chlorine, a free functional group other that those involved in the reaction being protected, if necessary, by a removable protecting group; or

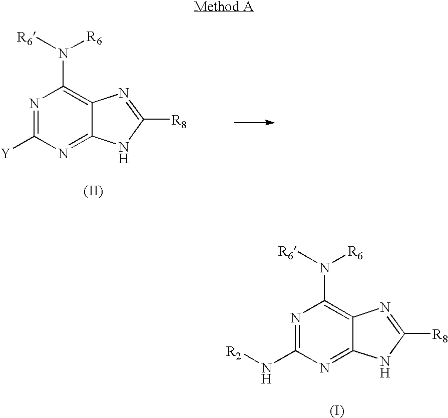

(B) reacting a substituted 9H-purin-6-yl of Formula (IV), substituted with a protecting group, R₉, with an hereroaryl/aryl-amine using preferably a palladium catalysed S_{N}Ar reaction and removing of the protecting group to form a compound of Formula (I), where R′₆, R₆, R₂, R₈, and Y are as defined above; or

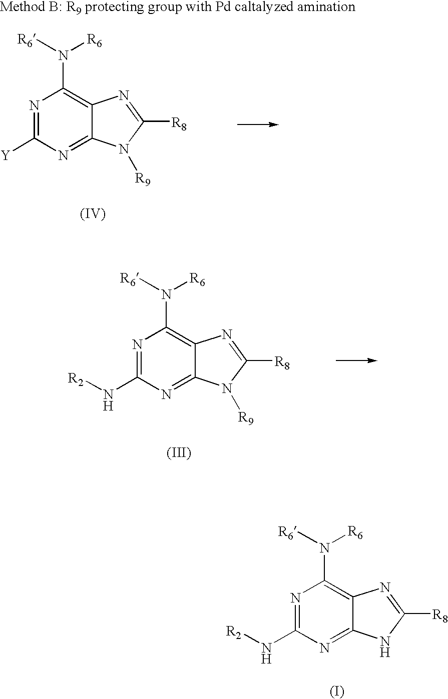

(C) reacting, in a solid phase, using a Rink acid resin, a substituted 9H-purin-6-yl, with an appropriate amine to afford substitution at the 6 position, followed by reaction with an heteroaryl/aryl-amine using preferably a palladium catalysed S_{N}Ar reaction to afford substitution at the 2 position, cleavage from the resin and purification, where R′₆, R₆, R₂, R₈, R₉ and Y are as defined above:

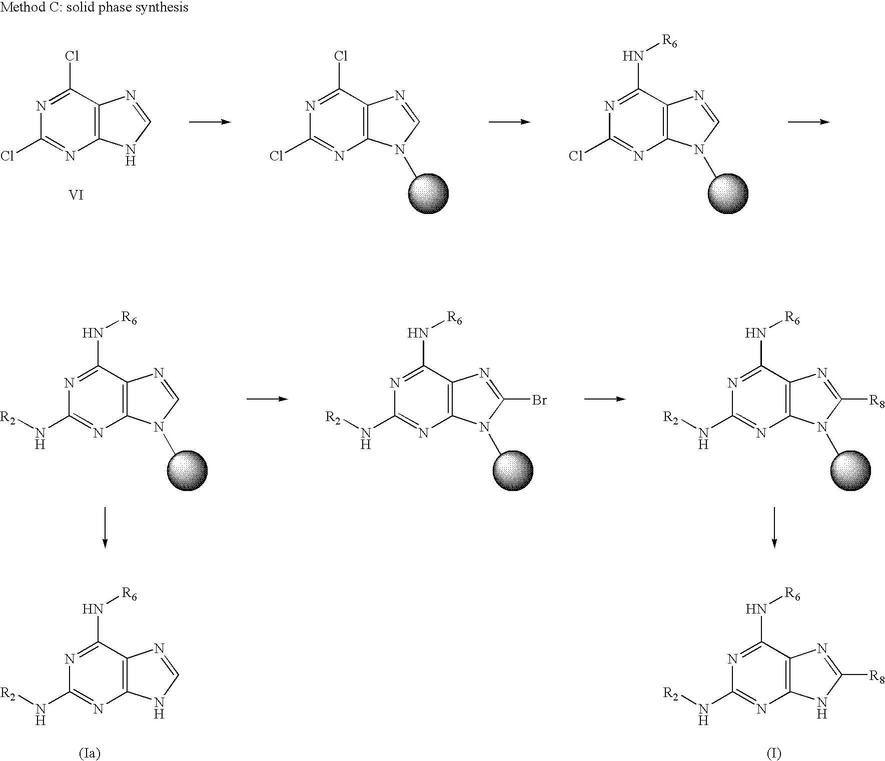

and, if desired, after reaction (A), (B) or (C), transforming an obtainable compound of Formula (I) into a different compound of Formula (I), or into a salt thereof, or vice versa from a salt to free compound, in a conventional manner; and/or separating an obtainable mixture of isomers of compounds of Formula (I) into the individual isomers; where for all reactions mentioned functional groups in the starting materials that shall not take part in the reaction are, if required, present in protected form by readily removable protecting groups, and any protecting groups are subsequently removed.

The compounds in free or salt form can be obtained in the form of hydrates or solvates containing a solvent used for crystallization.

Specifically, method (A) is performed in N-methyl-pyrrolidin-2-one at elevated temperatures, preferably between 100-150° C., or at 130° C., in the presence of a catalytic amount of HCl (0.1 eq.) for 18 to 120 h. The required product is isolated by (i) extraction from a 10% hydrogen carbonate solution and ethyl acetate, followed by flash silica chromatography or (ii) direct purification by preparative medium pressure liquid chromatography.

Method (B) C—N coupling reaction at position 2 is performed in presence of a catalytic amount of 2′-(dimethylamino)-2-biphenylyl-palladium(II) chloride dinorbornylphosphine complex in dry/degassed toluene at 110° C. with sodium tert-butylate as base. Deprotection is is performed in ethanol/water 5:1 and treated with an acid, preferably concentrated HCl at room temperature for up to six hours. The product is isolated by extraction from a 10% hydrogencarbonate solution and ethyl acetate, followed by flash silica chromatography.

Method (C) is performed on the solid phase with Rink acid resin to which is attached 2,6-dichloropurine followed by addition of an amine solution to obtain substitution at position 6. C—N coupling reaction at position 2 is performed in presence of a catalytic system including Pd₂(dba)₃/P(t-Bu)₃ in degassed NMP at 100° C. with CsCO₃ as base. The 8-position of the purine is brominated in the presence of the bromine-2,6-lutidine complex in NMP at RT protected from light. Heating of the resin for 20 h in degassed NMP in the presence of Pd(OAc)₂/CuO/1,3-bis-diphenylphosphino-propane with organo stannanes allows the introduction of R₈ in the 8-position of the purines. The resulting compounds are cleaved with TFA in 1,2-dichloroethane (20%) at RT followed by purification with an HPLC column from a Gilson 233XL. Separations are done by linear gradient elution of 5 min from 5% aqueous acetonitrile to 95% aqueous acetonitrile, both containing 0.1% trifluoroacetic acid. Samples are eluted on a 19×50 mm Waters Xterra 5μ column, using a flow rate of 20 ml/min. The target compounds are identified by electro spray ionization and collected by the automatic detection-before-collect routine. Fractions are collected using a Gilson 204 fraction collector accommodating 2 mega racks. The expected product from each sample present in the input rack is collected in one fraction (max. 8 mL, tarred glass tube 12×120 mm), based on mass detection, and placed at the same position in the output rack).

The following reaction conditions are preferred, respectively:

Within the scope of this text, only a readily removable group that is not a constituent of the particular desired end product of Formula (I) is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, and T. W. Greene and P. G. M. Wuts, “Protective Groups in Organic Synthesis”, Third edition, Wiley, New York 1999. A characteristic of protecting groups is that they can be removed readily (i.e. without the occurrence of undesired secondary reactions) for example by solvolysis, reduction, photolysis or alternatively under physiological conditions (e.g. by enzymatic cleavage).

The intermediates of Formula (IV) are prepared by following methods:

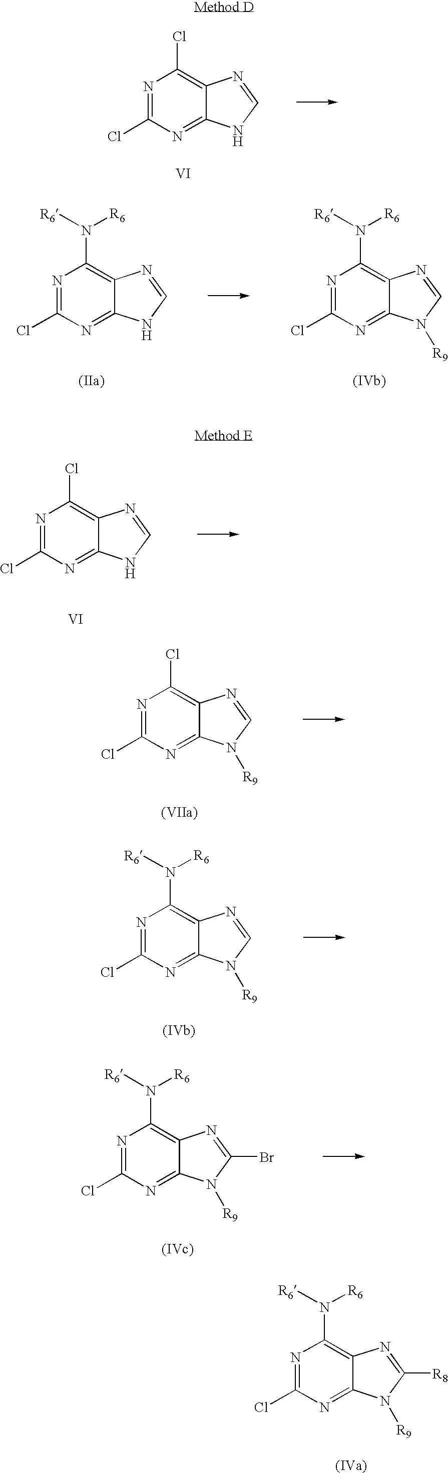

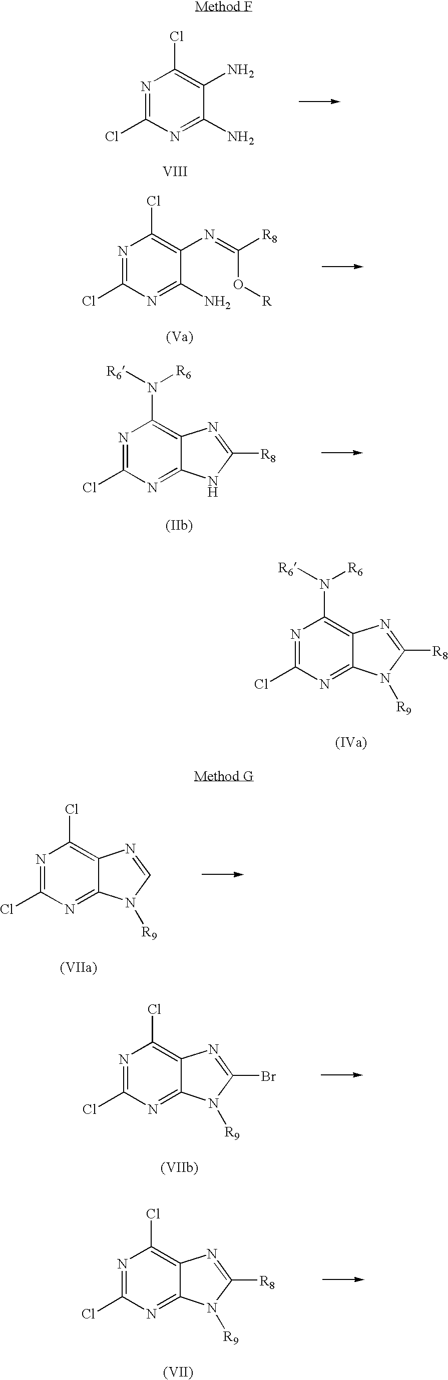

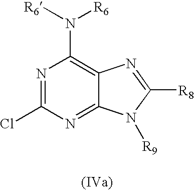

Salts of compound of Formula (I) can be prepared in a customary manner from the free compounds, and vice versa.

Mixtures of isomers obtainable according to the invention can be separated in a manner known per se into the individual isomers; diastereoisomers can be separated, for example, by partitioning between polyphasic solvent mixtures, recrystallization and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallization, or by chromatography over optically active column materials.

Intermediates and final products can be worked up and/or purified according to standard methods, e.g. using chromatographic methods, distribution methods, (re-) crystallization, and the like.

General Process Conditions

The following applies in general to all processes mentioned hereinbefore and hereinafter, while reaction conditions specifically mentioned above or below are preferred:

All the above-mentioned process steps can be carried out under reaction conditions that are known per se, preferably those mentioned specifically, in the absence or, customarily, in the presence of solvents or diluents, preferably solvents or diluents that are inert towards the re-agents used and dissolve them, in the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers as described above.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitrites, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

Pharmaceutical Compositions

The invention relates also to pharmaceutical compositions comprising a compound of Formula (I), to their use in the therapeutic (in a broader aspect of the invention also prophylactic) treatment or a method of treatment of proliferative disease, especially the preferred diseases mentioned above, to the compounds for said use and to the preparation of pharmaceutical preparations, especially for said uses.

The pharmacologically acceptable compounds of the present invention may be used, for example, for the preparation of pharmaceutical compositions that comprise an effective amount of a compound of the Formula (I), or a pharmaceutically acceptable salt thereof, as active ingredient together or in admixture with a significant amount of one or more inorganic or organic, solid or liquid, pharmaceutically acceptable carriers.

The invention relates also to a pharmaceutical composition that is suitable for administration to a warm-blooded animal, especially a human (or to cells or cell lines derived from a warm-blooded animal, especially a human, e.g. lymphocytes), for the treatment or, in a broader aspect of the invention, prevention of (=prophylaxis against) a disease that responds to inhibition of topoisomerase II activity, comprising an amount of a compound of Formula (I) or a pharmaceutically acceptable salt thereof, which is effective for said inhibition, especially the in, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to a method of treatment for a disease that responds to inhibition of topoisomerase II; which comprises administering an (against the mentioned disease) prophylactically or especially therapeutically effective amount of a compound of Formula (I) according to the invention, especially to a warm-blooded animal, for example a human, that, on account of one of the mentioned diseases, requires such treatment.

The dose of a compound of the Formula (I) or a pharmaceutically acceptable salt thereof to be administered to warm-blooded animals, for example humans of approximately 70 kg body weight, is preferably from approximately 3 mg to approximately 10 g, more preferably from approximately 10 mg to approximately 1.5 g, most preferably from about 100 mg to about 1000 mg/person/day, divided preferably into 1-3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dose form, such as in the form of ampoules, vials, suppositories, dragees, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C₈ to C₁₂, Hüls AG, Germany), but especially vegetable oils, such as cottonseed oil, almond oil, olive oil, castor oil, sesame oil, soybean oil and more especially groundnut oil.

The injection compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tricalcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient.

Combinations

The compounds of the present invention may be administered alone or in combination with other anticancer agents, such as other antiproliferative agents and compounds that inhibit tumor angiogenesis, for example, the protease inhibitors; epidermal growth factor receptor kinase inhibitors; vascular endothelial growth factor receptor kinase inhibitors and the like; cytotoxic drugs, such as antimetabolites, like purine and pyrimidine analog antimetabolites; antineoplastic antimetabolites; antimitotic agents like microtubule stabilizing drugs and antimitotic alkaloids; platinum coordination complexes; anti-tumor antibiotics; alkylating agents, such as nitrogen mustards and nitrosoureas; endocrine agents, such as adrenocorticosteroids, androgens, anti-androgens, estrogens, anti-estrogens, aromatase inhibitors, gonadotropin-releasing hormone agonists and somatostatin analogues and compounds that target an enzyme or receptor that is overexpressed and/or otherwise involved a specific metabolic pathway that is upregulated in the tumor cell, for example ATP and GTP phosphodiesterase inhibitors, histone deacetylase inhibitors, bisphosphonates; protein kinase inhibitors, such as serine, threonine and tyrosine kinase inhibitors, for example, Abelson protein tryosine kinase and the various growth factors, their receptors and kinase inhibitors therefore, such as, epidermal growth factor receptor kinase inhibitors, vascular endothelial growth factor receptor kinase inhibitors, fibroblast growth factor inhibitors, insulin-like growth factor receptor inhibitors and platelet-derived growth factor receptor kinase inhibitors and the like; compounds targeting, decreasing or inhibiting the activity of the AxI receptor tyrosine kinase family, the c-Met receptor or the Kit/SCFR receptor tyrosine kinase; methionine aminopeptidase inhibitors; matrix metalloproteinase inhibitors (“(MMP”); agents used in the treatment of hematologic malignancies; inhibitors of FMS-like tyrosine kinase receptors (Flt-3R); HSP-90 inhibitors; antiproliferative antibodies such as trastuzumab (Herceptin™), Trastuzumab-DM1, erlotinib (Tarceva™), bevacizumab (Avastin™), rituximab (Rituxan®), PRO64553 (anti-CD40) and 2C4 Antibody; antibodies such as intact monoclonal antibodies, polyclonal antibodies; further anti-angiogenic compounds such as thalidomide and TNP-470; compounds which target, decrease, or inhibit the activity of a protein or lipid phosphatase; compounds which induce cell differentiation processes; heparanase inhibitors; biological response modifiers; inhibitors of Ras oncogenic isoforms, e.g. farnesyl transferase inhibitors; telomerase inhibitors, methionine aminopeptidase inhibitors; proteasome inhibitors; and cyclooxygenase inhibitors, for example, cyclooxygenase-1 or -2 inhibitors. Also included are temozolomide, bengamides and m-Tor inhibitors. Most preferred are combinations of compounds of Formula (I) with ErbB-2 and HSP-90 inhibitors.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of the Formula (I), can be prepared and administered as described in the art such as in the documents cited above.

A compound of the Formula (I) may also be used to advantage in combination with known therapeutic processes, e.g., the administration of hormones or especially radiation.

A compound of Formula (I) may in particular be used as a radiosensitizer, especially for the treatment of tumors which exhibit poor sensitivity to radiotherapy.

The following examples serve to illustrate the invention without limiting the scope thereof:

### ABBREVIATIONS

dba dibenzylideneacetone

DCM dichloromethane

DMAP 4-dimethylaminopyridine

DMF dimethyl formamide

Et Ethyl

HCl hydrochloric acid

HPLC high pressure liquid chromatography

LDA lithium diisopropylamide

Me Methyl

m.p. melting point

MPLC medium pressure liquid chromatography

MS mass spectrometry

NMP N-Methyl-pyrrolidin-2-one

R_{f} retention factor

RT room temperature

TEAA triethylammonium acetate

TFA trifluoroacetic acid

TFAA trifluoroacetic acid anhydride

THF tetrahydrofuran

TLC thin layer chromatography

t_{R} retention time

Flash chromatography is performed by using silica gel (Merck 60). MPLC is performed with a Büchi system with reverse phase material Merck LiChroprep® RP-18. For thin layer chromatography, precoated silica gel (Merck 60 F254) plates are used. Detection of the components is made by UV light (254 nm). Without other indications HPLC analysis are performed on a Thermo Finnigan SpectraSYSTEM instrument, detector UV6000, column: 100×4.6 mm Chromolith Performance, RP-18e, solvent linear gradient from 2% B to 100% B over 8 min, then 2 min 100% B, flow rate 2.0 mL/min (Solvents: A=0.1% aqueous TFA and B=0.1% TFA in acetonitrile), retention time t_{R} given in minutes. Electrospray mass spectra are obtained with a Fisons Instruments VG Platform II. Melting points are measured with a Leica Galen III melting point apparatus. Commercially available solvents and chemicals are used for syntheses.

Where no temperatures are given, the reaction takes place at room temperature.

Ratios of solvents, e.g., in eluents or solvent mixtures, are given in volume by volume (v/v)

### EXAMPLES

Compounds of the present invention are synthesized according to the reactions steps summarized in Scheme 1:

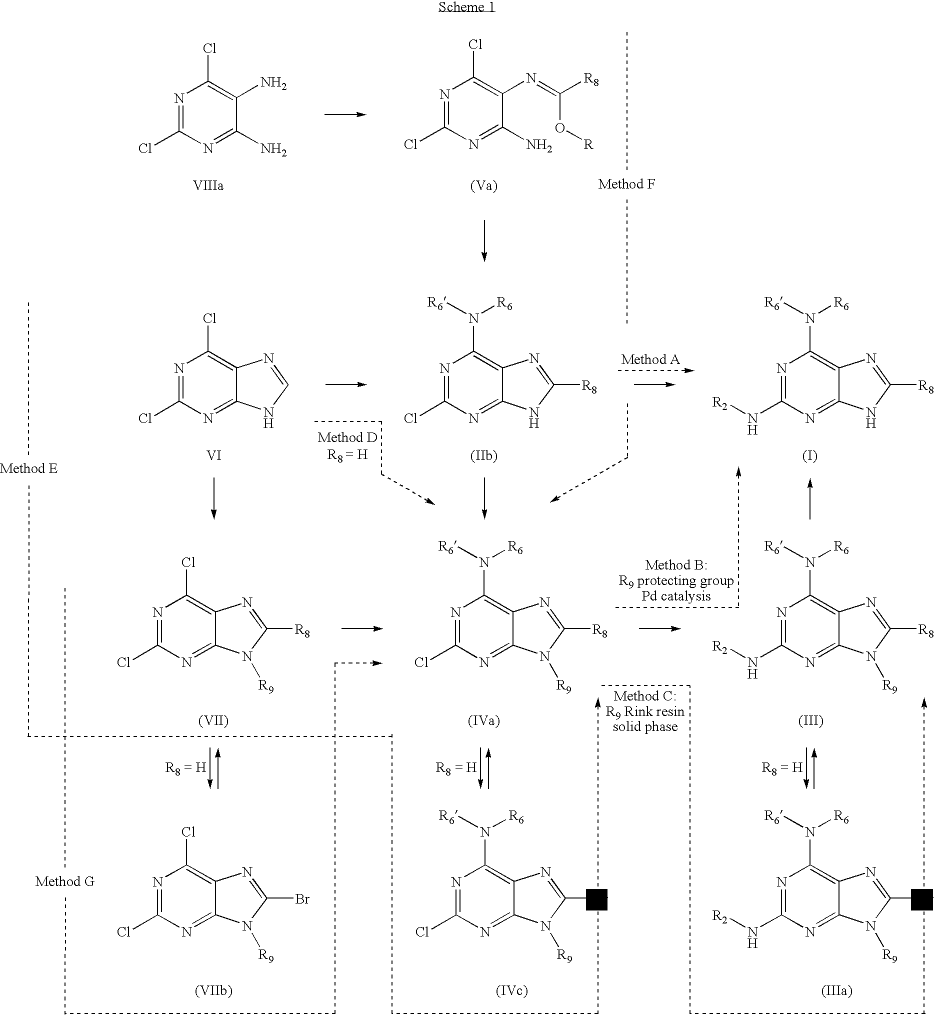

I. Synthesis of Intermediates

a): Steps 1.1, 4.1-12.1: Compounds of Formula (IIb) (Method D)

The intermediates shown in Table 1 are synthesized using the following procedure: A solution of 2,6-dichloro-9H-purine and the appropriate amine (2 eq.) are refluxed in ethanol for 4 to 18 h. The reaction mixture is cooled to RT, and the resulting product is isolated by extraction from a 10% hydrogen carbonate solution and ethyl acetate, followed by crystallization.

TABLE 1

| | |
|---|---|
| | (IIa) |
| 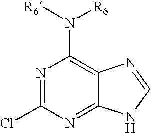 | |

Formula (IIa) where R′₆ = H

| EX | R₆ | Yield [%] | HPLC t_{R} | MS |
|---|---|---|---|---|
| 1.1 | Cycloheptyl- | 71 | 4.8 | 266 |
| 4.1 | 1-Hydroxy-2-methyl-prop-2-yl | 16 | 3.2 | 242 |
| 5.1 | Adamantan-2-yl- | 75 | 5.3 | 304 |
| 6.1ⁱ⁾ | Bicyclo[2.2.1]hept-2-yl- | 52 | 4.6 | 264 |
| 7.1 | Cyclooctyl- | 61 | 5.2 | 280 |
| 8.1ⁱⁱ⁾ | 3-Methoxy-phenyl- | 91 | 4.5 | 276 |
| 9.1 | 4-Hydroxy-cyclohexyl- | 47 | 2.8 | 268 |
| 10.1 | Quinolin-6-yl- | 24 | 2.7 | 297 |
| 11.1 | C,C-Diphenyl-methyl- | 54 | 5.5 | 336 |
| 12.1ⁱⁱⁱ⁾ | Phenyl- | 67 | 4.3 | 246 |

Previously described in ⁱ⁾WO 90/09178, ⁱⁱ⁾WO 97/16452 (Novartis), ⁱⁱⁱ⁾Nugiel, et al., J. Org. Chem. 1997, 62(1) 201-203.

b): Steps 13.1, 14.1-24.1 and 37.145.1: Compounds of Formula (III) (Method B)

The intermediates shown in Table 2 are synthesized starting with compounds of Steps 13.2, 14.2, 19.2, 23.2, 41.2, 42.3, 44.2 and 45.2 described below, using the following procedure for the palladium catalyzed amination:

A solution of the substituted [2-chloro-9-(tetrahydro-pyran-2-yl)-9H-purin-6-yl]-amine in dry/degassed toluene in added to sodium tert-butylate (1.4 eq.) in a dry flask kept under argon. The appropriate heteroaryl/aryl-amine (1.1 eq.) is added, the suspension is stirred for 20 min, heated to 110° C., and finally a solution of a catalytic amount of 2′-(dimethylamino)-2-biphenylyl-palladium(II) chloride dinorbornylphosphine complex (FLUKA, 0.02 eq.) in dry/degassed toluene is added. The reaction mixtures is stirred at 110° C. for 3 to 18 h, cooled to RT, and the required product is isolated by extraction from a 10% sodium bicarbonate solution and ethyl acetate, followed by (i) flash silica chromatography or (ii) crystallization.

For compounds of Steps 18.1, 21.1, 22.1, 23.1, and 37.1-40.1 the preparation of the aniline used as starting material is specifically described below.

TABLE 2

| | |
|---|---|
| | (III) |
| 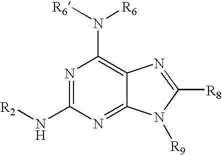 | |

Formula (III) where R′₆, R₈ = H and R₉ = tetrahydro-pyran-2-yl, exception *⁾R₉ = bis-(4-
methoxy-phenyl)-methyl

| | | | | Yield | HPLC | |
|---|---|---|---|---|---|---|
| EX | R₂ | R₆ | R₈ | [%] | t_{R} | MS |
| 13.1 | Quinolin-6-yl- | C,C-Diphenyl- | H | 68 | 5.03 | 528 |
| | | methyl- | | | | |
| 14.1 | Benzothiazol-6-yl- | tert-Butyl- | H | 38 | 5.03 | 424 |
| 15.1 | Naphthalen-2-yl- | tert-Butyl- | H | 48 | 6.02 | 417 |
| 16.1 | 2-Methyl-quinolin-6-yl- | tert-Butyl- | H | 50 | 4.24 | 432 |
| 17.1 | 2-Methyl-benzothiazol-5-yl- | tert-Butyl- | H | 100 | 5.16 | 438 |
| 18.1 | 2-tert-Butyl-benzothiazol-6- | tert-Butyl- | H | 55 | 6.26 | 480 |
| | yl- | | | | | |
| 19.1 | Quinolin-6-yl- | Cycloheptyl- | H | 49 | 4.68 | 458 |
| 20.1 | 4-Benzothiazol-2-yl-phenyl- | tert-Butyl- | H | 89 | 6.79 | 500 |
| 21.1 | 6-Methoxy-naphthalen-2-yl- | tert-Butyl- | H | 79 | 5.9 | 447 |
| 22.1 | 6-(2-Morpholin-4-yl-ethoxy)- | tert-Butyl- | H | 31 | 4.3 | 546 |
| | naphthalen-2-yl- | | | | | |
| 23.1*⁾ | 1-BOC-Indol-5-yl- | tert-Butyl- | H | 56 | 7.41 | 661.8 |
| 24.1*⁾ | 1-BOC-Indol-5-yl- | tert-Butyl- | H | 25 | 7.19 | 647.9 |
| 37.1*⁾ | 2-(2-Morpholin-4-yl-ethoxy)- | tert-Butyl- | Ethyl- | 44 | 4.91 | 723.2 |
| | benzothiazol-6-yl- | | | | | |
| 37.3 | 2-(2-Morpholin-4-yl-ethoxy)- | tert-Butyl- | Ethyl- | 24 | 11.7ⁱ⁾ | 497.3 |
| | benzothiazol-6-yl- | | | | | |
| 38.1 | 2-(2-Morpholin-4-yl-ethoxy)- | tert-Butyl- | Isopropyl- | 25 | 12.5ⁱ⁾ | 511.1 |
| | benzothiazol-6-yl- | | | | | |
| 39.1 | 2-(2-Morpholin-4-yl-ethoxy)- | tert-Butyl- | Cyclopropyl- | 46 | 12.9ⁱ⁾ | 509.3 |
| | benzothiazol-6-yl- | | | | | |
| 40.1 | 2-(2-Morpholin-4-yl-ethoxy)- | tert-Butyl- | Cyclopentyl- | 20 | 13.4ⁱ⁾ | 537.3 |
| | benzothiazol-6-yl- | | | | | |
| 41.1 | Benzothiazol-6-yl- | tert-Butyl- | Cyclopropyl- | 53 | 5.28 | 464.2 |
| 42.1 | Benzothiazol-6-yl- | tert-Butyl- | 2,4-Dimethoxy- | 34 | 5.97 | 589.2 |
| | | | benzylamino- | | | |
| 43.1 | Naphthalen-2-yl- | tert-Butyl- | 2,4-Dimethoxy- | 55 | 6.81 | 582.3 |
| | | | benzylamino- | | | |
| 44.1 | Naphthalen-2-yl- | tert-Butyl- | (2,4-Dimethoxy- | 47 | 5.973 | 596.2 |
| | | | benzyl)-methyl- | | | |
| | | | amino- | | | |
| 45.1 | Benzothiazol-6-yl- | tert-Butyl- | 1-Ethoxyvinyl- | 20 | 2.10ⁱⁱ⁾ | 494 |

ⁱ⁾HPLC conditions: Agilent 1100 instrument, C18BDS (4.6 × 250 mm) SC/355 column; 20 mM NH₄OAc/acetonitrile 3:2 for 5 min, up to acetonitrile during 5 min, then acetonitrile
ⁱⁱ⁾HPLC conditions: Thermo Finnigan SpectraSYSTEM instrument, detector UV2000, column: 50 × 4.6 mm Chromolith SpeedROD, RP-18e, solvent linear gradient from 2% B to 100% B over 2.5 min, then 0.5 min 100% B, flow rate 4.0 mL/min (Solvents: A = 0.1% aqueous formic acid and B = 0.1% formic acid in acetonitrile)

c) Steps 18.2, 21.2, 22.2 23.4, and 37.5: Preparation of the Anilines

### Step 18.2: 2-tert-Butyl-benzothiazol-6-ylamine

Prepared According Ciba-Geigy CH 565164 19720815.

### Step 21.2: 6-methoxy-naphthalen-2-ylamine

A solution of 2-bromo-6-methoxy-naphthalene (237 mg, 1 mmol), Pd(dba)₂ (28 mg, 0.05 mmol), tri-t-buthylphosphonium tetrafluoroborate (14 mg, 0.05 mmol), and lithium bis(trimethylsilyl)amide (1 M in hexane, 1.1 mL, 1.1 mmol) is stirred in toluene (2.5 mL) under argon for 6 h. The reaction mixture is taken in diethyl ether (20 mL), and quenched with 1 M HCl. The organic phase is extracted with water, the combined aqueous phases are treated with 1 M sodium hydroxide and extracted with DCM. The combined organic phases are dried over magnesium sulfate, the solvent is evaporated in vacuo and the residue is purified by column flash chromatography on silica gel (ethyl acetate) to afford 6-methoxy-naphthalen-2-ylamine (110 mg, 64%), HPLC t_{R}: 3.07, (M+H)+=173.9.

### Step 22.2: 6-(2-morpholin-4-yl-ethoxy)-naphthalen-2-ylamine

A solution of 6-bromo-naphthalen-2-ol (2.2 g, 10 mmol), 4-(2-chloro-ethyl)-morpholine hydrochloride (1.9 g, 10 mmol), potassium carbonate (2.9 g, 21 mmol) are dissolved in DMF (100 mL) and stirred for 18 h at 60° C. The reaction mixture is cooled to RT, diluted with ethyl acetate, and washed with brine. The combined organic phases are dried over sodium sulfate, the solvent is evaporated in vacuo to afford 4-[2-(6-bromo-naphthalen-2-yloxy)-ethyl]-morpholine (3.18, 94%), HPLC t_{R}: 3.95, (M+H)+=336. Subsequently, the obtained 4-[2-(6-bromo-naphthalen-2-yloxy)-ethyl]-morpholine is reacted using an analogous procedure as described for Step 21.2 to afford 6-(2-morpholin-4-yl-ethoxy)-naphthalen-2-ylamine. Yield 27%, HPLC t_{R}: 2.04, (M+H)+=273.1.

### Step 23.4: 5-amino-2-methyl-indole-1-carboxylic acid tert-butyl ester

A solution of 2-methyl-5-nitro-1H-indole (7.0 g, 40 mmol), BOC-anhydride (17.5 g, 80 mmol), and DMAP (733 mg, 6 mmol) in dioxane (250 ml) are stirred for 15 min at RT. The solvent is evaporated in vacuo and the residue is purified by column flash chromatography on silica gel (hexane/ethyl acetate 1:1) to afford 2-methyl-5-nitro-indole-1-carboxylic acid tert-butyl ester (9.2 g, 83%), HPLC t_{R}: 6.89, (M+H)+=277. Subsequently, a solution of the obtained 2-methyl-5-nitro-indole-1-carboxylic acid tert-butyl ester (9.1 g, 33 mmol) in ethyl acetate (250 ml) is hydrogenated over Pd(C) 10% (1 g) at RT and normal pressure. The reaction mixture is filtered over Celite®, the solvent is evaporated in vacuo, the residue is purified by column flash chromatography on silica gel (hexane/ethyl acetate 4:1 to 6:4) to afford 5-amino-2-methyl-indole-1-carboxylic acid tert-butyl ester (5.8 g, 70%), HPLC t_{R}: 4.15, (M+H)+=247.1.

### Step 37.5: 2-(2-morpholin-4-yl-ethoxy)-benzothiazol-6-ylamine

A solution of 2-morpholin-4-yl-ethanol (328 mg, 2.5 mmol) in THF (10 mL) stirred at 0° C. is added sodium hydride (55%, 120 mg, 2.75 mmol), followed by 2-chloro-6-nitro-benzothiazole (537 mg, 2.5 mmol). The reaction mixture is stirred for 1 h at RT, extracted with ethyl acetate, washed with brine, the combined organic phases are dried over sodium sulfate, the solvent is evaporated in vacuo to afford crude 2-(2-morpholin-4-yl-ethoxy)-6-nitro-benzothiazole (700 mg, 90%), HPLC t_{R}: 3.36, (M+H)+=310. Subsequently, a solution of 2-(2-morpholin-4-yl-ethoxy)-6-nitro-benzothiazole (680 mg, 2.2 mmol) in ethanol (50 ml) is hydrogenated over Pd(C) 10% (0.1 g) at RT and normal pressure. The reaction mixture is filtered over Celite®, the solvent is evaporated in vacuo, the residue is purified by column flash chromatography on silica gel (DCM/methanol 19:1) to afford 2-(2-morpholin-4-yl-ethoxy)-benzothiazol-6-ylamine (582 mg, 94%), HPLC t_{R}: 1.35, (M+H)+=280.

d): Steps 13.2, 14.2, 19.2, 23.2, 37.2-39.2, 40.2-45.2

### Step 13.2: benzhydryl-[2-chloro-9-(tetrahydro-pyran-2-yl)-9H-purin-6-yl]-amine (Method D)

Benzhydryl-(2-chloro-9H-purin-6-yl)-amine (Step 11.1, 800 mg, 2.6 mmol) is stirred vigorously with a catalytic amount of p-toluenesulfonic acid (4.5 mg, 0.03 mmol) in ethyl acetate (10 mL) at 55° C. Subsequently, a solution of 3,4-dihydro-2H-pyran (0.38 mL, 5.2 mmol) in ethyl acetate (1 mL) is added drop wise. After 1 h 30 at 55° C. more 3,4-dihydro-2H-pyran (0.14 mL, 2.6 mmol) is added and the reaction mixture is stirred for 1 h at 55° C., cooled to RT, neutralized with 10% sodium bicarbonate, extracted with ethyl acetate, the combined organic phases are dried over sodium sulfate, the solvent is evaporated in vacuo and the residue is purified by column flash chromatography on silica gel (ethyl acetate/hexane 2:1) to afford benzhydryl-[2-chloro-9-(tetrahydro-pyran-2-yl)-9H-purin-6-yl]-amine (1.0 g, 91%), TLC R_{f} (Silica gel, hexane/ethyl acetate 1:1): 0.5,HPLC t_{R}: 6.7, (M+H)+=420.

### Step 14.2: tert-butyl-[2-chloro-9-(tetrahydro-pyran-2-yl)-9H-purin-6-yl]-amine (Method E)

A suspension of 2,6-dichloro-9-(tetrahydro-pyran-2-yl)-9H-purine [Cassidy et al., Journal of Heterocyclic Chemistry 1968, 5(4), 461-465] (546 mg, 2 mmol) and tert-butylamine (2.1 mL, 20 mmol) in ethanol (10 mL) is refluxed for 2 h 30, cooled to RT, neutralized with 10% sodium bicarbonate, extracted with ethyl acetate, the combined organic phases are dried over sodium sulfate, the solvent is evaporated in vacuo and the residue is purified by column flash chromatography on silica gel (ethyl acetate/hexane 1:1) to afford tert-butyl-[2-chloro-9-(tetrahydro-pyran-2-yl)-9H-purin-6-yl]-amine (575 mg, 93%), TLC R_{f} (Silica gel, hexane/ethyl acetate 1:1): 0.4, HPLC t_{R}: 5.9, (M+H)+=310.

### Step 19.2: [2-Chloro-9-(tetrahydro-pyran-2-yl)-9H-purin-6-yl]-cycloheptyl-amine (Method D)

Compound of Step 19.2 is synthesized using an analogous procedure as described in Step 13.2 (THF as solvent). Yield 74%, TLC R_{f} (Silica gel, hexane/ethyl acetate 1:1): 0.4, HPLC t_{R}: 6.6, (M+H)+=350.

### Step 23.2: {9-[bis-(4-methoxy-phenyl)-methyl]-2-chloro-9H-purin-6-yl}-tert-butyl-amine (Method E)

To a solution of 2,6-dichloro-9H-purine (4.1 g, 25 mmol) and bis-(4-methoxy-phenyl)-methanol (5.8 g, 22.5 mmol) in acetic acid (100 mL) is added concentrated sulfuric acid (0.13 mL, 2.5 mmol). The reaction mixture is stirred for 2 h at RT, diluted with water (150 ml), the precipitate is filtered, washed with water and dried in vacuo at 60° C to afford 9-[bis-(4-methoxy-phenyl)-methyl]-2,6-dichloro-9H-purine (9.6 g, 92%). Subsequently, this material (9.1 g, 22 mol) is dissolved in ethanol (50 mL) with t-butylamine (11.5 ml, 110 mmol), and the solution is stirred for 3 h at 60° C. in a closed pressure safe vial. The reaction mixture is cooled to RT, diluted with ethyl acetate, extracted with a saturated sodium bicarbonate solution, the combined organic phases are dried over sodium sulfate, the solvent is evaporated in vacuo and the residue is purified by column flash chromatography on silica gel (ethyl acetate/hexane 1:4) to afford {9-[bis-(4-methoxy-phenyl)-methyl]-2-chloro-9H-purin-6-yl}-tert-butyl-amine (6.1 g, 61%), HPLC t_{R}: 6.75, (M+H)+=451.9.

### Step 37.2: {9-[bis-(4-methoxy-phenyl)-methyl]-2-chloro-8-ethyl-9H-purin-6-yl}-tert-butyl-amine (Method F)

To a solution of tert-butyl-(2-chloro-8-ethyl-9H-purin-6-yl)-amine (compound of Step 27.1, 710 mg, 2.8 mmol) and bis-(4-methoxy-phenyl)-methanol (615 mg, 2.52 mmol) in acetic acid (10 mL) is added concentrated sulfuric acid (0.015 mL, 0.28 mmol). The reaction mixture is stirred for 18 h at 50° C., diluted with ice cold water (150 ml), neutralized with 10% sodium bicarbonate, extracted with DCM, the combined organic phases are dried over sodium sulfate, the solvent is evaporated in vacuo and the residue is purified by column flash chromatography on silica gel (ethyl acetate/hexane 1:4) to afford {9-[bis-(4-methoxy-phenyl)-methyl]-2-chloro-8-ethyl-9H-purin-6-yl}-tert-butyl-amine (900 mg, 67%), HPLC t_{R}: 7.03, (M+H)+=480.4.

Compounds shown in Table 3 are synthesized using an analogous procedure as described in Step 13.2 starting from Steps 27.1, 33.1, 34.1 and 41.5 described below (Method F).

TABLE 3

| | |
|---|---|
| | (IVa) |
| 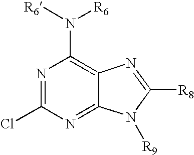 | |

Formula (IVa) where R′₆ = H and R₉ = tetrahydro-pyran-2-yl

| Ex. | R₆ | R₈ | Yield [%] | HPLC t_{R}ⁱ⁾ | MS |
|---|---|---|---|---|---|
| 37.4 | tert-Butyl- | Ethyl- | 90 | 4.79 | 338 |
| 38.2 | tert-Butyl- | Isopropyl- | 77 | 4.90 | 352 |
| 39.2 | tert-Butyl- | Cyclopropyl- | 87 | 4.83 | 350 |
| 40.2 | tert-Butyl- | Cyclopentyl- | 75 | 5.35 | 378 |

ⁱ⁾HPLC conditions: Agilent 1100 instrument, C18BDS (4.6 × 250 mm) SC/355 column; 0.1% Trifluoroacetic acid/acetonitrile Flow: 0.8 mL/min

### Step 41.2 (Alternate Synthesis of Compound of Step 39.2: tert-butyl-[2-chloro-8-cyclopropyl-9-(tetrahydro-pyran-2-yl)-9H-purin-6-yl]-amine (Method E)

To a solution of diisopropylamine (0.43 mL, 3 mmol) in dry THF (13 mL) stirred at −78° C. is added a solution of 1.6 M butyllithium in hexane (1.9 mL, 3 mmol). The solution was is briefly stirred at 0° C., and cooled again to −78° C. (standard procedure for obtaining a fresh solution of LDA). tert-Butyl-[2-chloro-9-(tetrahydro-pyran-2-yl)-9H-purin-6-yl]-amine (compound of Step 14.2, 310 mg, 1 mmol) in THF (2 mL) is then added dropwise over 10 min to the LDA solution stirred at −78° C. The reaction mixture is stirred for 1 h at the same temperature, and cyanogen bromide (328 mg, 3 mmol) in THF (1 mL) is added dropwise over 10 min to the reaction mixture which is further stirred for 1 h. The reaction mixture is quenched with a 10% ammonium chloride solution, allowed to reach 0° C., diluted with ethyl acetate, extracted with 10% ammonium chloride solution, and brine. The combined organic phases are dried over sodium sulfate, the solvent is evaporated in vacuo and the residue is purified by column flash chromatography on silica gel (CombiFlash® Companion) RediSep® column hexane/ethyl acetate gradient 20:1 to 10:3) to afford [8-Bromo-2-chloro-9-(tetrahydro-pyran-2-yl)-9H-purin-6-yl]-tert-butyl-amine (389 mg, quant.), HPLC t_{R}: 6.63, (M+H)+=390. Subsequently, the obtained [8-Bromo-2-chloro-9-(tetrahydro-pyran-2-yl)-9H-purin-6-yl]-tert-butyl-amine (77 mg, 0.2 mmol), cyclopropylboronic acid (26 mg, 0.3 mmol), potassium phosphate tribasic (127 mg, 0.6 mmol), and [1,1′-bis(diphenylphosphino)ferrocene]dichloropalladium(II) complex with DCM (1.6 mg, 0.002 mmol) are stirred in dioxane (1 mL) under argon for 18 h at 100° C. The reaction mixture is cooled to RT, diluted with ethyl acetate, washed with 10% sodium bicarbonate, and brine. The combined organic phases are dried over sodium sulfate, the solvent is evaporated in vacuo and the residue is purified by column flash chromatography on silica gel (CombiFlash® Companion® RediSep® column hexane/ethyl acetate gradient 20:1 to 10:3) to afford tert-butyl-[2-chloro-8-cyclopropyl-9-(tetrahydro-pyran-2-yl)-9H-purin-6-yl]-amine (35 mg, 50%), HPLC t_{R}: 6.25, (M+H)+=350.1.

### Step 42.3: N*6*-tert-butyl-2-chloro-N*8*-(2,4-dimethoxy-benzyl)-9-(tetrahydro-pyran-2-yl)-9H-purine-6,8-diamine (Buchwald Type Amination, Method E)

A solution of [8-Bromo-2-chloro-9-(tetrahydro-pyran-2-yl)-9H-purin-6-yl]-tert-butyl-amine (compound of Step 41.2, 77 mg, 0.2 mmol), 2,4-dimethoxybenzylamine (40 mg, 0.24 mmol), potassium phosphate tribasic (60 mg, 0.28 mmol), biphenyl-2-yl-di-tert-butyl-phosphane (8 mg, 0.02 mmol), and Pd₂(dba)₃ (9 mg, 0.02 mmol) are stirred in dimethoxyethane (0.5 mL) under argon for 20 h at 100° C. The reaction mixture is cooled to RT, diluted with ethyl acetate, washed with 10% sodium bicarbonate, and brine. The combined organic phases are dried over sodium sulfate, the solvent is evaporated in vacuo and the residue is purified by column flash chromatography on silica gel (CombiFlash® Companion® RediSep® column hexane/ethyl acetate gradient 20:1 to 4:1) to afford N*6*-tert-butyl-2-chloro-N*8*-(2,4-dimethoxy-benzyl)-9-(tetrahydro-pyran-2-yl)-9H-purine-6,8-diamine (55 mg, 58%), HPLC t_{R}: 6.03, (M+H)+=475.1.

### Step 44.2: N*6*-tert-Butyl-2-chloro-N*8*-(2,4-dimethoxy-benzyl)-N*8*-methyl-9-(tetrahydro-pyran-2-yl)-9H-purine-6,8-diamine (Method E)

Compound of Step 44.2 is synthesized with (2,4-dimethoxy-benzyl)-methyl-amine using an analogous procedure as described in Step 42.3 ((2′-Dicyclohexylphosphanyl-biphenyl-2-yl)-dimethyl-amine as phosphine ligand). Yield 54%, HPLC t_{R}: 4.92, (M+H)+=489.

### Step 45.2 tert-butyl-[2-chloro-8-(1-ethoxy-vinyl)-9-(tetrahydro-pyran-2-yl)-9H-purin-6-yl]-amine (Stille Coupling, Method G)

The bromination of 2,6-dichloro-9-(tetrahydro-pyran-2-yl)-9H-purine is done using an analogous procedure a described for Step 41.2, with 1,2-dibrom-tetrachlorethane as the bromide source, to afford 8-bromo-2,6-dichloro-9-(tetrahydro-pyran-2-yl)-9H-purine. Yield 82%, HPLC t_{R}: 5.22, (M-Tetrahydropyrane)+=266.9. Subsequently, a solution of the obtained 8-bromo-2,6-dichloro-9-(tetrahydro-pyran-2-yl)-9H-purine (176 mg, 0.5 mmol), tributyl-(1-ethoxy-vinyl)-stannane (217 mg, 0.6 mmol), Pd₂(dba)₃ DCM complex (26 mg, 0.05 mmol), and tri(2-furyl)phosphine (6 mg, 0.05 mmol) in DMF (5 mL) is stirred at 50° C. under argon for 1 h. The reaction mixture is cooled at RT, the solvent is evaporated in vacuo, the residue is dissolved in acetonitrile, washed with hexane, the acetonitrile is removed in vacuo, and the residue is purified by column flash chromatography on silica gel (CombiFlash® Companion® RediSep® column hexane/ethyl acetate gradient 20:1 to 4:1) to afford 2,6-dichloro-8-(1-ethoxy-vinyl)-9-(tetrahydro-pyran-2-yl)-9H-purine (91 mg, 53%), HPLC t_{R} (conditions see Table 2ⁱⁱ⁾): 1.84, (M+H)+=343. In a further step, using a procedure tert-butyl-[2-chloro-8-(1-ethoxy-vinyl)-9-(tetrahydro-pyran-2-yl)-9H-purin-6-yl]-amine is obtained using an analogous as described in Step 14.2. Yield 54%, HPLC t_{R} (conditions see Table 2ⁱⁱ⁾): 2.13, (M+H)+=296.

e): Steps 25.1-34.1 and 41.5: Compounds of Formula (IIb) (Method F)

Compounds shown in Table 4 are synthesized from Steps 25.2, 27.2, 32.2, 33.2, 34.2 and 39.3 using one of the two following procedure:

the acetimidic acid ester dissolved in the appropriate alkyl amine and NMP as co-solvent (4:1 ratio) is heated for 2 h at 150° C. using a microwave (Emrys optimizer 300 W). Extraction from a 10% hydrogen carbonate solution and ethyl acetate afforded pure product without further purification.

a solution of the substituted acetimidic acid ester and the appropriate alkylamine (10 eq.) in butanol were heated for 40 h to 8 days at 100-140° C. The required product is isolated by extraction from a 10% sodium bicarbonate solution and ethyl acetate, followed by (i) flash silica chromatography or (ii) crystallization.

TABLE 4

| | |
|---|---|
| | (IIb) |
| 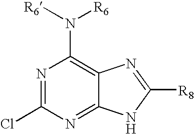 | |

Formula (IIb) where R′₆ = H, *⁾N—R₆ R′₆ = heterocyclic amine

| Ex. | R₆ | R₈ | Yield [%] | HPLC t_{R} | MS |
|---|---|---|---|---|---|
| 25.1 | tert-Butyl- | Methyl- | 58.4 | 4.04 | 240 |
| 26.1 | Cycloheptyl- | Methyl- | 67 | 4.74 | 280 |
| 27.1 | tert-Butyl- | Ethyl- | 45 | 4.18 | 254 |
| 28.1 | 2,2,2-Trifluoro-1- | Ethyl- | 29 | 4.22 | 294 |
| | methyl-ethyl- | | | | |
| 29.1 | 2-Methyl-pyrrolidin- | Ethyl- | 5 | 3.82 | 266 |
| | 1-yl*⁾ | | | | |
| 30.1 | 3-yl-Butyramide | Ethyl- | 12 | 2.68 | 283 |
| 31.1 | 3-yl-Butyric acid | Ethyl- | 66 | 3.14 | 312 |
| | ethyl ester | | | | |
| 32.1 | tert-Butyl- | Propyl- | 25 | 3.5 | 268 |
| 33.1 | tert-Butyl- | Isopropyl- | 69 | 3.01ⁱ⁾ | 268 |
| 34.1 | tert-Butyl- | Cyclopentyl- | 56 | 3.30ⁱ⁾ | 294 |
| 41.5 | tert-Butyl- | Cyclopropyl- | 78 | 2.96ⁱ⁾ | 266 |

ⁱ⁾HPLC conditions: Agilent 1100 instrument, C18BDS (4.6 × 250 mm) SC/340 column; 20 mM NH₄OAc/acetonitrile 3:2 for 5 min, up to acetonitrile during 5 min, then acetonitrile

f: Steps 25.2, 27.2, 32.2, 33.2, 34.2 and 39.3: Compounds of Formula (Va) (Method F):

The intermediates, Steps 25.2, 27.2 and 32.2, shown in Table 5 are synthesized using the following procedure:

A solution of 2,6-dichloro-pyrimidine-4,5-diamine [Legravend, et al., Synthesis 1990, 587-589] in the appropriate triethyl or trimethyl orthoester is heated for 45 min. at 100° C. The reaction mixture is cooled to RT, diethyl ether is added, the precipitate is filtered off, and washed with diethyl ether.

Compound of Steps 33.2, 34.2 and 39.3 (Table 5) are synthesized using the following procedure:

A solution of 2,6-dichloro-pyrimidine-4,5-diamine [Legravend, et al., Synthesis 1990, 587-589] and the appropriate carboximidic acid methyl ester [gained from the corresponding nitrile, DeWolfe, et al. Synthesis, 1974, 153-172] in dry methanol is heated for 24 h at 60° C. The reaction mixture is cooled to RT and the solvent is removed under reduced pressure. The desired product is isolated by flash silica chromatography.

TABLE 5

| | |
|---|---|
| | (Va) |
| 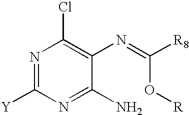 | |

Formula (Va) where Y = Cl

| EX | R₈ | R | Yield [%] | HPLC t_{R} | MS |
|---|---|---|---|---|---|
| 25.2 | Methyl- | Ethyl- | 72 | 4.41 | 251 |
| 27.2 | Ethyl- | Ethyl- | 78 | 4.94 | 263 |
| 32.2 | Propyl- | Methyl- | 52 | 4.4 | 264 |
| 33.2 | Isopropyl- | Methyl- | 10 | 3.69ⁱ⁾ | 263 |
| 34.2 | Cyclopentyl- | Methyl- | 25 | 4.12ⁱ⁾ | 289 |
| 39.3 | Cyclopropyl- | Methyl- | 77 | 3.19ⁱ⁾ | 261 |

ⁱ⁾HPLC conditions: Agilent 1100 instrument, C18BDS (4.6 × 250 mm) SC/340 column; 0.1% Trifluoroacetic acid/acetonitrile Flow: 0.8 mL/min

### II. Examples 1-74

### a): Examples 1-13 and 25-36: Compound of Formula (I) (Method A)

The compounds from Examples 1-13 and 25-36 shown in Table 6 are synthesized from the compound of Steps 1.1, 4.1-12.1, 25.1-34.1 and 41.5 using the following procedure:

A solution of the above mentioned substituted 2-chloro-9H-purin-6-ylamine and the appropriate heteroaryl/aryl-amine (1-2 eq.) in NMP is heated at 130° C. in presence of a catalytic amount of HCl (0.1 eq.) for 18 to 120 h. The product is isolated by (i) extraction from a 10% hydrogen carbonate solution and ethyl acetate, followed by flash silica chromatography or (ii) direct purification by preparative MPLC.

### b): Examples 13-24 and 37-45: Compound of Formula (I) (Method B)

The compounds from Examples 13-24 and 37-45 shown in Table 6 are prepared from the protected purines Steps 13.1-24.1 and 37.1-45.1 using the following procedures for the deprotection:

For Examples 13-22, 36, 38-41 and 45, a solution of the above mentioned 9-(tetrahydro-pyran-2-yl)-9H-purine in ethanol/water 5:1 is treated with concentrated HCl (30 eq.) at RT for 1 to 6 h. The product is isolated by extraction from a 10% sodium bicarbonate solution and ethyl acetate, followed by flash silica chromatography.

For Examples 23, 24, 37, and 42-44, the above mentioned protected purine is treated with a solution of TFA/DCM 1:1 at RT for 4 to 18 h. The product is isolated by extraction from a 10% sodium bicarbonate solution and ethyl acetate, followed by flash silica chromatography.

### c): Examples 46-74: Compound of Formula (I) (Method C)

The compounds from Examples 46-74 shown in Table 6 are synthesized on solid phase using the following procedure:

Preparation of the solid phase: the Rink acid resin (Nova Biochem, loading: 0.6 mmol/g, 70-90 mesh) is washed thoroughly before use (10× dioxane, 5× DCM, 10× DMF, 10× dioxane/water 1:1, 5× ethanol, 5× dioxane, 5× alternating with DCM and methanol, 5× alternating with DCM and pentane, 3× pentane) and dried (40° C., 0.25 bar, overnight).

Attachment to the Solid Phase: the Rink resin (10 g) is placed into a flame dried reaction vessel. TFAA (15 mL in 80 ml 2,6-lutidine) is added. After standing for 10 min, the resin is filtered off, TFAA (15 mL in 80 ml 2,6-lutidine) is added and shaken for 2 h at RT. The resin is filtered off, washed with DCM (2×100 mL, DCM is filtered over Alox prior to use). 2,6-Dichloropurine (5.7 g dissolved in 55 mL NMP) is added, filtered after 10 min. A second portion of 2,6-dichloropurine (5.7 g dissolved in 55 mL NMP) is added and the reaction mixture shaken for 18 h at RT. The resin is filtered off, washed (5× NMP, 5× DMSO, 5× alternating with DCM and methanol, 5× alternating with DCM and pentane, 3× DCM) and dried (40° C., 0.25 bar, overnight). A suspension of the resin in toluene/DCE is distributed into mini-Kans® (IRORI) by using a matrix multipipette (100 mg resin per Kan). Each Kan is equipped with a radiofrequency transponder, sealed and dried.

Substitution at the 6-position: the Kans are distributed into the corresponding reaction bottles (500 mL). An amine solution (2M) in NMP is added (2 mL per Kan, corresponds to 30 equiv.). The solutions are flushed with argon. After standing for 5 d at 55° C. the Kans are washed (5× DMF, 5× TEAA in water/DMF (1:4), 5× DMF, 5× acetic acid/DCM (20%), 5× DCM, 5× alternating with DCM and methanol, 5× alternating with DCM and pentane, 5× DCM) and dried.

Substitution at the 2-position; the Kans are distributed into the reaction bottles (500 mL, one bottle per building block). Cs₂CO₃ is added (35 mg per Kan), the bottles are purged with argon. Pd₂(dba)₃ (6 mg per Kan) is added, followed by the amines (1 M solution in NMP, 2 mL per Kan, corresponds to 30 equiv.). The solutions are degassed by placing the bottles in an ultrasound bath and passing argon through the solution during 15 min. P(t-Bu)₃ (0.016 ml per Kan) is transferred into the bottles (manipulations in atmosbag), the bottles are sealed and heated to 100° C. for 7 d. The Kans are then washed (5× DMF, 5× TEAA in water/DMF (1:4), 5× DMF, 5× water, 5× DCM, 5× alternating with DCM and methanol, 5× alternating with DCM and pentane) and dried.

Bromination of the 8-position: The Kans are distributed into bottles. NMP (1 mL per Kan) and a solution of Br₂.2,6-lutidine (0.6 g per Kan) and 2,6-lutidine (0.03 mL per Kan) in NMP (1 mL per Kan) are added. The bottles are shaken for 4 h under argon at RT and protected from light. The Kans are washed with (3× DCM and NMP) and dried. The bromination step is repeated three times as described previously.

Stille coupling at the 8-position: The Kans are distributed into the reaction bottle and flushed with argon. NMP (2 mL per Kan), CuO (30 mg per Kan) and Pd(OAc)₂ (8.2 mg per Kan) are added, the solution is degassed and purged with argon. 1,3-Bis-diphenylphosphino-propane (30.8 mg per Kan) and the organo stannane is added (manipulations under argon; in atmosbag). The bottles are sealed. After standing for 20 h at 100-105° C., the Kans were washed (5× water, 3× DMF, 2× DCM, 5× AcOH/MeCN/water 2:5:3,5 alternatingly with DCM and MeOH, 2× pentane) and dried.

Cleavage from Resin: the Kans are distributed into the cleavage tubes, the compounds are cleaved with TFA in 1,2-dichloroethane (20%) during 4 h at RT, the solutions collected into tubes.

Purification: the solution of the tubes are evaporated, the samples, dissolved in 500 μL DMA, and automatically injected into the prep. HPLC column from a Gilson 233XL. Separations are done by linear gradient elution of 5 min from 5% aqueous acetonitrile to 95% aqueous acetonitrile, both containing 0.1% TFA. Samples are eluted on a 19×50 mm Waters Xterra 5μ column, using a flow rate of 20 mL/min. The target compounds are identified by electro spray ionization and collected by the automatic detection-before-collect routine. Fractions are collected using a Gilson 204 fraction collector accommodating 2 mega racks. The expected product from each sample present in the input rack is collected in one fraction (max. 8 mL, tarred glass tube 12×120 mm), based on mass detection, and placed at the same position in the output rack).

TABLE 6

| | |
|---|---|
| | Formula (I) |
| 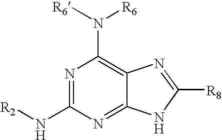 | |

Formula (I) R′₆ = H, *⁾N—R₆ R′₆ = heterocyclic amine

| | | | | | Yield | HPLC | | TOPO II |
|---|---|---|---|---|---|---|---|---|
| Ex. | Method | R₂ | R₆ | R₈ | [%] | t_{R} | MS | inh^{.i)} |
| 1 | A | Benzothiazol-6-yl- | Cycloheptyl- | H | 69 | 4.6 | 380 | + |
| 2 | A | 4-Thiazol-2-yl- | Cycloheptyl- | H | 13 | 5.0 | 406 | − |
| | | phenyl | | | | | | |
| 3 | A | Quinolin-6-yl- | Cycloheptyl- | H | 77 | 3.6 | 374 | − |
| 4 | A | Benzothiazol-6-yl- | 1-Hydroxy-2- | H | 30 | 3.3 | 356 | − |
| | | | methyl-prop-2-yl | | | | | |
| 5 | A | Benzothiazol-6-yl- | Adamantan-2-yl- | H | 74 | 4.9 | 418 | + |
| 6 | A | Benzothiazol-6-yl- | Bicyclo[2.2.1]hept- | H | 60 | 4.3 | 378 | + |
| | | | 2-yl- | | | | | |
| 7 | A | Benzothiazol-6-yl- | Cyclooctyl- | H | 74 | 4.7 | 394 | + |
| 8 | A | Benzothiazol-6-yl- | 3-Methoxy- | H | 21 | 4.0 | 390 | − |
| | | | phenyl- | | | | | |
| 9 | A | Benzothiazol-6-yl- | 4-Hydroxy- | H | 24 | 3.3 | 382 | − |
| | | | cyclohexyl- | | | | | |
| 10 | A | Quinolin-6-yl- | Quinolin-6-yl- | H | 10 | 2.9 | 405 | − |
| 11 | A | Benzothiazol-6-yl- | C,C-Diphenyl- | H | 14 | 4.6 | 450 | − |
| | | | methyl- | | | | | |
| 12 | A | Quinolin-6-yl- | Phenyl- | H | 21 | 3.4 | 354 | + |
| 13 | A/B | Quinolin-6-yl- | C,C-Diphenyl- | H | 2/54 | 3.9 | 444 | − |
| | | | methyl- | | | | | |
| 14ⁱⁱ⁾ | B | Benzothiazol-6-yl- | tert-Butyl- | H | 57 | 3.9 | 340 | ++ |
| 15 | B | Naphthalen-2-yl- | tert-Butyl- | H | 73 | 4.9 | 333 | ++ |
| 16 | B | 2-Methyl-quinolin- | tert-Butyl- | H | 78 | 3.2 | 348 | + |
| | | 6-yl- | | | | | | |
| 17 | B | 2-Methyl- | tert-Butyl- | H | 40 | 4.2 | 354 | − |
| | | benzothiazol-5-yl- | | | | | | |
| 18 | B | 2-tert-Butyl- | tert-Butyl- | H | 59 | 5.0 | 396 | ++ |
| | | benzothiazol-6-yl- | | | | | | |
| 19 | B | 2-Methyl- | tert-Butyl- | H | 54 | 4.0 | 354 | + |
| | | benzothiazol-6-yl- | | | | | | |
| 20 | B | 4-Benzothiazol-2- | tert-Butyl- | H | 46 | 5.4 | 416 | ++ |
| | | yl-phenyl | | | | | | |
| 21 | B | 6-Methoxy- | tert-Butyl- | H | 77 | 4.8 | 363.1 | +++ |
| | | naphthalen-2-yl- | | | | | | |
| 22 | B | 6-(2-Morpholin-4-yl- | tert-Butyl- | H | 20 | 3.6 | 462 | +++ |
| | | ethoxy)- | | | | | | |
| | | naphthalen-2-yl- | | | | | | |
| 23 | B | 2-Methyl-1H-indol- | tert-Butyl- | H | 60 | 4.3 | 336 | − |
| | | 5-yl- | | | | | | |
| 24 | B | 1H-Indol-5-yl- | tert-Butyl- | H | 11 | 4.1 | 322 | − |
| 25 | A | Benzothiazol-6-yl- | tert-Butyl- | Methyl- | 30 | 4.0 | 354 | ++ |
| 26 | A | Benzothiazol-6-yl- | Cycloheptyl- | Methyl- | 39 | 4.4 | 394 | − |
| 27 | A | Benzothiazol-6-yl- | tert-Butyl- | Ethyl- | 22 | 4.2 | 368 | +++ |
| 28 | A | Benzothiazol-6-yl- | 2,2,2-Trifluoro-1- | Ethyl- | 39 | 4.3 | 408 | + |
| | | | methyl-ethyl- | | | | | |
| 29 | A | Benzothiazol-6-yl- | 2-Methyl- | Ethyl- | 64 | 4.2 | 380 | − |
| | | | pyrrolidin-1-yl-*⁾ | | | | | |
| 30 | A | Benzothiazol-6-yl- | 3-yl-Butyramide | Ethyl- | 11 | 4.0 | 397 | 0 |
| 31 | A | Benzothiazol-6-yl- | 3-yl-Butyric acid | Ethyl- | 6 | 4.1 | 426 | − |
| | | | ethyl ester | | | | | |
| 32 | A | Benzothiazol-6-yl- | tert-Butyl- | Propyl- | 6 | 4.11 | 382 | − |
| 33 | D | Benzothiazol-6-yl- | tert-Butyl- | Isopropyl- | 55 | 2.8ⁱⁱⁱ⁾ | 382.0 | − |
| 34 | D | Benzothiazol-6-yl- | tert-Butyl- | Cyclopentyl- | 58 | 3.1ⁱⁱⁱ⁾ | 408.0 | − |
| 35 | A | 2-Oxo-2H- | tert-Butyl- | Ethyl- | 55 | 4.2 | 379 | ++ |
| | | chromen-6-yl- | | | | | | |
| 36 | A | 2-Methylsulfanyl- | tert-Butyl- | Ethyl- | 56 | 4.9 | 414 | +++ |
| | | benzothiazol-6-yl- | | | | | | |
| 37 | B | 2-(2-Morpholin-4-yl- | tert-Butyl- | Ethyl- | 81 | 3.4 | 497 | +++ |
| | | ethoxy)- | | | | | | |
| | | benzothiazol-6-yl- | | | | | | |
| 38 | E | 2-(2-Morpholin-4-yl- | tert-Butyl- | Isopropyl- | 25 | 12.5ⁱⁱⁱ⁾ | 511.1 | − |
| | | ethoxy)- | | | | | | |
| | | benzothiazol-6-yl- | | | | | | |
| 39 | E | 2-(2-Morpholin-4-yl- | tert-Butyl- | Cyclopropyl- | 46 | 12.9ⁱⁱⁱ⁾ | 509.3 | ++ |
| | | ethoxy)- | | | | | | |
| | | benzothiazol-6-yl- | | | | | | |
| 40 | E | 2-(2-Morpholin-4-yl- | tert-Butyl- | Cyclopentyl- | 20 | 13.4ⁱⁱⁱ⁾ | 537.3 | 0 |
| | | ethoxy)- | | | | | | |
| | | benzothiazol-6-yl- | | | | | | |
| 41 | B | Benzothiazol-6-yl- | tert-Butyl- | Cyclopropyl- | 89 | 4.3 | 380.2 | ++ |
| 42 | B | Benzothiazol-6-yl- | tert-Butyl- | —NH₂ | 74 | 4.08 | 355.2 | ++ |
| 43 | B | Naphthalen-2-yl- | tert-Butyl- | —NH₂ | 40 | 4.9 | 348 | ++ |
| 44 | B | Naphthalen-2-yl- | tert-Butyl- | Methyl- | 83 | 5.06 | 362.2 | ++ |
| | | | | amino- | | | | |
| 45 | B | Benzothiazol-6-yl- | tert-Butyl- | Acetyl- | 100 | 4.3 | 382 | − |
| 46 | C | Quinolin-6-yl- | tert-Butyl- | H | 100 | 3.5^{iv)} | 334.4 | + |
| 47 | C | Benzo[1,2,5]thiadia- | tert-Butyl- | H | 69 | 4.6^{iv)} | 341.4 | − |
| | | zol-5-yl- | | | | | | |
| 48 | C | 2-Methyl- | tert-Butyl- | H | 96 | 4.2^{iv)} | 354.5 | − |
| | | benzothiazol-6-yl- | | | | | | |
| 49 | C | Benzo[1,2,5]thiadia- | Cycloheptyl- | H | 98 | 5.5^{iv)} | 381.5 | − |
| | | zol-5-yl- | | | | | | |
| 50 | C | 2-Methyl- | Cycloheptyl- | H | 80 | 4.8^{iv)} | 394.5 | + |
| | | benzothiazol-6-yl- | | | | | | |
| 51 | C | Benzothiazol-6-yl- | 1,1-Dimethyl- | H | 97 | 4.2^{iv)} | 354.5 | + |
| | | | propyl- | | | | | |
| 52 | C | Quinolin-6-yl- | 1,1-Dimethyl- | H | 78 | 3.6^{iv)} | 348.4 | − |
| | | | propyl- | | | | | |
| 53 | C | Benzo[1,2,5]thiadia- | 1,1-Dimethyl- | H | 82 | 4.8^{iv)} | 355.4 | − |
| | | zol-5-yl- | propyl- | | | | | |
| 54 | C | 2-Methyl- | 1,1-Dimethyl- | H | 80 | 4.4^{iv)} | 368.5 | − |
| | | benzothiazol-6-yl- | propyl- | | | | | |
| 55 | C | Benzothiazol-6-yl- | 2-(4-Fluoro- | H | 100 | 4.7^{iv)} | 434.5 | − |
| | | | phenyl)-1,1- | | | | | |
| | | | dimethyl-ethyl- | | | | | |
| 56 | C | Quinolin-6-yl- | 2-(4-Fluoro- | H | 85 | 4.0^{iv)} | 428.5 | − |
| | | | phenyl)-1,1- | | | | | |
| | | | dimethyl-ethyl- | | | | | |
| 57 | C | Benzo[1,2,5]thiadia- | 2-(4-Fluoro- | H | 97 | 5.3^{iv)} | 435.5 | − |
| | | zol-5-yl- | phenyl)-1,1- | | | | | |
| | | | dimethyl-ethyl- | | | | | |
| 58 | C | 2-Methyl- | 2-(4-Fluoro | H | 95 | 4.8^{iv)} | 448.5 | − |
| | | benzothiazol-6-yl- | phenyl)-1,1- | | | | | |
| | | | dimethyl-ethyl- | | | | | |
| 59 | C | Benzothiazol-6-yl- | 4-Pyridin-3-yl- | H | 96 | 3.4^{iv)} | 430.5 | − |
| | | | piperazin-1-yl*⁾ | | | | | |
| 60 | C | Benzothiazol-6-yl- | 4-Pyridin-2-yl- | H | 96 | 3.4^{iv)} | 430.5 | − |
| | | | piperazin-1-yl-*⁾ | | | | | |
| 61 | C | Quinolin-6-yl- | 4-Pyridin-2-yl- | H | 82 | 3.2^{iv)} | 424.5 | − |
| | | | piperazin-1-yl-*⁾ | | | | | |
| 62 | C | Benzo[1,2,5]thiadia- | 4-Pyridin-2-yl- | H | 84 | 3.8^{iv)} | 431.5 | − |
| | | zol-5-yl- | piperazin-1-yl-*⁾ | | | | | |
| 63 | C | 2-Methyl- | 4-Pyridin-2-yl- | H | 80 | 3.6^{iv)} | 444.5 | − |
| | | benzothiazol-6-yl- | piperazin-1-yl-*⁾ | | | | | |
| 64 | C | Quinolin-6-yl- | 2,3,5,6- | H | 85 | 3.5^{iv)} | 425.5 | − |
| | | | tetrahydro- | | | | | |
| | | | [1,2′]bipyrazinyl- | | | | | |
| | | | 4-yl-*⁾ | | | | | |
| 65 | C | Benzothiazol-6-yl- | tert-Butyl- | H | 100 | 4.1^{iv)} | 340.4 | + |
| 66 | C | Benzothiazol-6-yl- | Cycloheptyl- | H | 95 | 4.9^{iv)} | 380.5 | − |
| 67 | C | Quinolin-6-yl- | Cycloheptyl- | H | 97 | 4.0^{iv)} | 374.5 | − |
| 68 | C | Benzothiazol-6-yl- | 1-Hydroxy-2- | H | 56 | 3.4^{iv)} | 356.4 | − |
| | | | methyl-prop-2-y-l | | | | | |
| 69 | C | Naphthalen-2-yl- | tert-Butyl- | Br | 50 | 4.97^{iv)} | 411.3 | + |
| 70 | C | Naphthalen-2-yl- | tert-Butyl- | Vinyl- | 15 | 5.00^{iv)} | 358.5 | − |
| 71 | C | Naphthalen-2-yl- | tert-Butyl- | Allyl- | 15 | 5.15^{iv)} | 372.5 | − |
| 72 | C | Naphthalen-2-yl- | tert-Butyl- | Methyl- | 8 | 4.78^{iv)} | 346.4 | − |
| 73 | C | Naphthalen-2-yl- | tert-Butyl- | Ethyl- | 15 | 4.04^{iv)} | 362 | − |
| 74 | C | Naphthalen-2-yl- | tert-Butyl- | iso-Butyl- | 17 | 4.53^{iv)} | 389 | − |

ⁱ⁾TOPO II inhibition corresponding to the inhibition of the TOPO ATPase activity in the above described
Malachite Green assay

| | | | | |
|---|---|---|---|---|
| Percent inhibition | | <80% | <65% | |
| @10 μM | ≧80% | ≧65% | ≧50% | <50% |
| Symbol | +++ | ++ | + | − |

ⁱⁱ⁾Previously described in WO 2001/009134 (Novartis)
ⁱⁱⁱ⁾HPLC conditions: Agilent 1100 instrument, C18BDS (4.6 × 250 mm) SC/340 column; 20 mM NH₄OAc/
acetonitrile 3:2 for 5 min, up to acetonitrile during 5 min, then acetonitrile
^{iv)}HPLC column from a Gilson 233XL. Separations are done by linear gradient elution of 5 min from
5% aqueous acetonitrile to 95% aqueous acetonitrile, both containing 0.1% TFA.
Samples are eluted on a 19 × 50 mm Waters Xterra 5 μ column, using a flow rate of 20 m/min.
MS: The target compounds are identified by electro spray ionization and collected by the automatic detection-
before-collect routine.
ⁱ⁾HPLC conditions: Agilent 1100 instrument, C18BDS (4.6 × 250 mm) SC/340 column; 20 mM NH₄OAc/
acetonitrile 3:2 for 5 min, up to acetonitrile during 5 min, then acetonitrile

### Example 75

### Tablets 1 Comprising Compounds of the Formula (I)

Tablets, comprising, as active ingredient, 50 mg of any one of the compounds of Formula (I) mentioned in the preceding Examples 1-74 of the following composition are prepared using routine methods:

| Composition: | | |
|---|---|---|
| Active Ingredient | 50 | mg |
| Wheat starch | 60 | mg |
| Lactose | 50 | mg |
| Colloidal silica | 5 | mg |
| Talcum | 9 | mg |
| Magnesium stearate | 1 | mg |
| | 175 | mg |

Manufacture: The active ingredient is combined with part of the wheat starch, the lactose and the colloidal silica and the mixture pressed through a sieve. A further part of the wheat starch is mixed with the 5-fold amount of water on a water bath to form a paste and the mixture made first is kneaded with this paste until a weakly plastic mass is formed.

The dry granules are pressed through a sieve having a mesh size of 3 mm, mixed with a pre-sieved mixture (1 mm sieve) of the remaining corn starch, magnesium stearate and talcum and compressed to form slightly biconvex tablets.

### Example 76

### Tablets 2 Comprising Compounds of the Formula (I)

Tablets, comprising, as active ingredient, 100 mg of any one of the compounds of Formula (I) of Examples 1-74 are prepared with the following composition, following standard procedures:

| Composition: | | |
|---|---|---|
| Active Ingredient | 100 | mg |
| Crystalline lactose | 240 | mg |
| Avicel | 80 | mg |
| PVPPXL | 20 | mg |
| Aerosil | 2 | mg |
| Magnesium stearate | 5 | mg |
| | 447 | mg |

Manufacture: The active ingredient is mixed with the carrier materials and compressed by means of a tabletting machine (Korsch EKO, Stempeldurchmesser 10 mm).

### Example 77

### Capsules

Capsules, comprising, as active ingredient, 100 mg of any one of the compounds of Formula (I) given in Examples 1-74, of the following composition are prepared according to standard procedures:

| Composition: | | |
|---|---|---|
| Active Ingredient | 100 | mg |
| Avicel | 200 | mg |
| PVPPXL | 15 | mg |
| Aerosil | 2 | mg |
| Magnesium stearate | 1.5 | mg |
| | 318.5 | mg |

Manufacturing is done by mixing the components and filling them into hard gelatine capsules, size 1.

### Example 78

### ATP Competitive Inhibitor Activity

The below chart shows that a compound according to Example 1 is an ATP competitive inhibitor. OD refers to Optical Density measured at 630 nm and is measured spectrophotometrically.

[image]

### Example 79

### Gain of Selectivity by Decrease Kinase Inhibitory Activity Using Position 8 Substituted 9H-Purine-2,6-Diamine Derivatives

Activity determinations of compounds of the preceding examples, using the testing method described in the references below, with the following test compounds of Formula (I) exhibit activity for the following kinases shown in the table below

### Percentage Inhibition of the Kinase at 10 μM Concentration of the Inhibitor

| | Kinase pannel inh. @ 10 μM | | | | | | | | | | | | | | |
|---|---|---|---|---|---|---|---|---|---|---|---|---|---|---|---|
| | ABL | FGFR | Flt-1 | Flt-4 | HER1 | HER2 | IGF | KDR | KIT | MET | PDK1 | PKA | PKB | Raf-1 | Tek |
| 14 | +++ | ++ | ++ | +++ | ++ | ++ | +++ | +++ | +++ | +++ | +++ | +++ | + | ++ | +++ |
| 25 | ++ | − | − | − | − | − | − | + | − | − | − | + | − | − | − |
| 27 | + | − | − | − | − | − | − | + | − | − | − | − | − | − | ++ |

Percent inhibition @ 10 μM

| | ≧80% | <80% ≧ 65% | <65% ≧ 50% | <50% |
|---|---|---|---|---|
| Symbol | +++ | ++ | + | − |

### REFERENCES FOR THE KINASE ASSAYS

Paul W. Manley, Pascal Furet, Guido Bold, Josef Brüggen, Jürgen Mestan, Thomas Meyer, Christian R. Schnell, and Jeanette Wood; Anthranilic Acid Amides: A Novel Class of Antiangiogenic VEGF Receptor Kinase Inhibitors J. Med. Chem. 2002, 45, 5687-5693. Wan, Yongqin; Hur, Wooyoung; Cho, Charles Y.; Liu, Yi; Adrian, Francisco J.; Lozach, Olivier; Bach, Stephane; Mayer, Thomas; Fabbro, Doriano; Meijer, Laurent; Gray, Nathanael S; Synthesis and Target Identification of Hymenialdisine Analogs Chemistry & Biology 2004, 11(2), 247-259.

1. A method of treating a proliferative disease comprising administering a compound of the Formula (I) 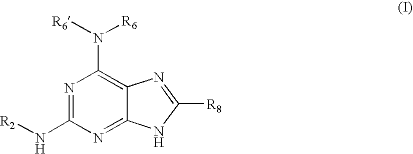
wherein:
R₂ is substituted or unsubstituted aryl, substituted or unsubstituted bicyclic aryl, substituted or unsubstituted heteroaryl, substituted or unsubstituted bicyclic heteroaryl;
R′₆ is H or lower alkyl;
R₈ is H, halo, lower alkyl, lower alkenyl, small cycloalkyl, acetyl, —NR₁₂R₁₃ where R₁₂ and R₁₃ are independently H or lower alkyl;
R₆ is substituted or unsubstituted aryl, substituted or unsubstituted heteroaryl, substituted or unsubstituted bicyclic aryl, substituted or unsubstituted bicyclic heteroaryl, or a substituted or unsubstituted aliphatic residue; or R₆ and R′₆ with the N atom form a substituted or unsubstituted heterocyclic radical;
or pharmaceutically acceptable salts thereof,
to a warm-blooded animal, especially a human, in need of such treatment: or
pharmaceutically acceptable salts thereof.

2. A method according to claim 1, wherein the proliferative disease is a benign or malignant tumor, a carcinoma of the brain, kidney, liver, adrenal gland, bladder, breast, stomach, gastric tumors, ovaries, colon, rectum, prostate, pancreas, lung, vagina, thyroid, sarcoma, glioblastomas, multiple myeloma or gastrointestinal cancer, colon carcinoma or colorectal adenoma, or a tumor of the neck and head, an epidermal hyperproliferation, prostate hyperplasia, a neoplasia, or a leukemia.

3. A method according to claim 1 wherein the proliferative disease is selected from cancers and tumors with low levels of topoisomerase II.

4. A compound of Formula (I): 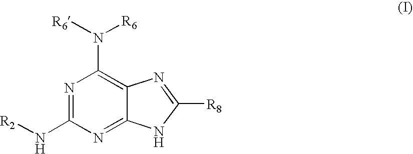
wherein:
R₂ is substituted or unsubstituted aryl, substituted or unsubstituted bicyclic aryl, substituted or unsubstituted heteroaryl, substituted or unsubstituted bicyclic heteroaryl;
R′₆ is H or lower alkyl;
R₈ is lower alkyl or small cycloalkyl;
R₆ is substituted or unsubstituted aryl, substituted or unsubstituted heteroaryl, substituted or unsubstituted bicyclic aryl, substituted or unsubstituted bicyclic heteroaryl, or a substituted or unsubstituted aliphatic residue; or R₆ and R′₆ with the N atom form a substituted or unsubstituted heterocyclic radical;
or pharmaceutically acceptable salts thereof.

5. A compound of Formula (I) according to claim 4 wherein:
R₂ is phenyl; phenyl substituted with thiazolyl; benzothiazolyl; benzothioazoyl substituted with lower alkyl such as methyl or t-butyl or substituted with lower alkyl sulfanyl such as methyl sulfanyl; quinolinyl; quinolinyl substituted with methyl; naphthyl; indolyl; benzo[1,2,5]thiadiazolyl; chromenyl; chromen-2-one or amino chromen-2-one; and
R₆ is cycloheptyl; cyclooctyl; cycloheptyl; cyclohexyl or cyclohexyl substituted with hydroxy; adamantanyl; bicyclo[2.2.1] heptyl; phenyl or phenyl substituted with lower alkoxy, e.g. methoxy; quinolinyl; lower alkyl such as t-butyl; 2,2,2-trifluoro-1-methyl-ethyl-; methyl or methyl substituted with diphenyl; ethyl or ethyl substituted with methyl and fluorophenyl, e.g. 2-(fluoro-phenyl)-1,1-dimethyl-ethyl; propyl or propyl substituted with methyl or hydroxy e.g. 1,1-dimethyl propyl or 1-hydroxy-2-methyl-prop-2-yl; lower aliphatic ester e.g. 3-yl-butyric acid ethyl ester or amide 3-yl-butyramide;
R₆R′₆N is piperazinyl substituted with pyridine or pyrazine; or pyrrolidin-1-yl e.g. 2-methyl-pyrrolidin-1-yl;
or pharmaceutically acceptable salts thereof.

6. A compound according to claim 4 wherein R₂ is aryl or heteroaryl substituted with R′₂ where R′₂ is H or a solubilizing group of the Formula:
—X—Y-A
where X is O, S, —(CH₂)ₙ—, NH or N(lower alkyl);
Y is —(CH₂)ₙ;
n is 1-4, preferably 2-3; and
A is NR₁₀R₁₁ where R₁₀ and R₁₁ are independently H or C₁-C₃ lower alky, such as methyl, ethyl or propyl, or R₁₀ and R₁₁ with the nitrogen atom form a 3- to 8-membered heterocyclic ring containing 1-4 nitrogen, oxygen or sulfur atoms (e.g. morpholinyl, piperazinyl or lower alkyl-piperazinyl) or A is 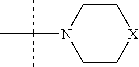
where X is as defined above.

7. A compound according to claim 6 wherein R₂ is selected from

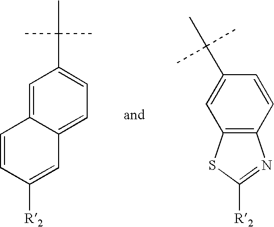

8. A compound according to claims 1 wherein R₆ is bicyclic alkyl, tricyclic alkyl, or heteroaryl, all of which may be substituted or unsubstituted.

9. A compound according to Formula (II): 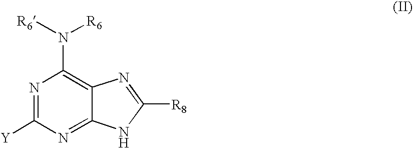
R′₆ is H or lower alkyl; R₈ is H, halo or lower alkyl; small cycloalkyl, and
R₆ is substituted or unsubstituted aryl, substituted or unsubstituted bicyclic aryl or a substituted or unsubstituted aliphatic residue, or a substituted or unsubstituted aliphatic ester or amide
or R₆ and R′₆ with the N form a heterocyclic radical;
Y is a protecting group selected from chlorine, bromine or iodine
or pharmaceutically acceptable salts thereof, with the proviso that if R₈ is H, then R′₆ cannot be bicyclo[2.2.1]hept-2-ylamine, methoxyphenyl or phenyl.

10. A compound of Formula (III): 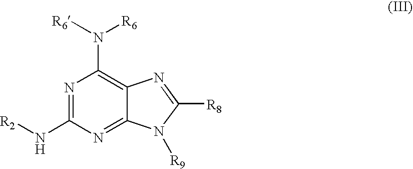
wherein:
R₂ is substituted or unsubstituted aryl, substituted or unsubstituted bicyclic aryl, substituted or unsubstituted heteroaryl, substituted or unsubstituted bicyclic heteroaryl;
R′₆ is H or lower alkyl;
R₈ is H, halo, lower alkyl, lower alkenyl, small cycloalkyl, acetyl, —NR₁₂R₁₃ where R₁₂ and R₁₃ are independently H or lower alkyl;
R₉ is a protecting group;
R₆ is substituted or unsubstituted aryl, substituted or unsubstituted bicyclic aryl or a substituted or unsubstituted aliphatic residue,
or R₆ and R′₆ with the N form a heterocyclic radical;
or pharmaceutically acceptable salts thereof.

11. A compound of Formula (IV): 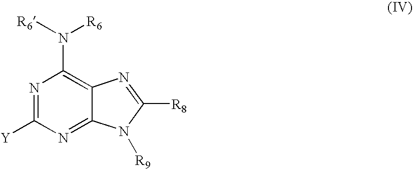
wherein:
R′₆ and R₈ are each independently H, halo, or lower alkyl;
R₉ is a protecting group;
Y is a protecting group selected from chlorine, bromine or iodine;
R₆ is substituted or unsubstituted aryl, substituted or unsubstituted heteroaryl, substituted or unsubstituted bicyclic aryl, substituted or unsubstituted bicyclic heteroaryl, or a substituted or unsubstituted aliphatic residue; or R₆ and R′₆ with the N atom form a substituted or unsubstituted heterocyclic radical;
or pharmaceutically acceptable salts thereof.

12. A compound of Formula (V): 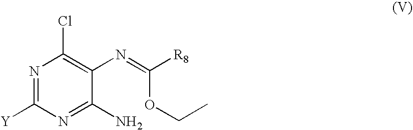
R₈ is H, halo or lower alkyl and Y is a protecting group selected from chlorine, bromine or iodine.

13. A pharmaceutical composition comprising a compound according to claim 4.

14. A pharmaceutical composition comprising a compound according to claim 4 and an acceptable pharmaceutical carrier.

15. A compound according to claim 1 selected from the group consisting of:
N*2*-Benzothiazol-6-yl-N*6*-cycloheptyl-9H-purine-2,6-diamine;
N*6*-Cycloheptyl-N*2*-(4-thiazol-2-yl-phenyl)-9H-purine-2,6-diamine;
N*6*-Cycloheptyl-N*2*-quinolin-6-yl-9H-purine-2,6-diamine;
2-[2-(Benzothiazol-6-ylamino)-9H-purin-6-ylamino]-2-methyl-propan-1-ol;
N*6*-Adamantan-2-yl-N*2*-benzothiazol-6-yl-9H-purine-2,6-diamine;
N*2*-Benzothiazol-6-yl-N*6*-bicyclo[2.2.1]hept-2-yl-9H-purine-2,6-diamine;
N*2*-Benzothiazol-6-yl-N*6*-cyclooctyl-9H-purine-2,6-diamine;
N*2*-Benzothiazol-6-yl-N*6*-(3-methoxy-phenyl)-9H-purine-2,6-diamine;
4-[2-(Benzothiazol-6-ylamino)-9H-purin-6-ylamino]-cyclohexanol;
N*2*,N*6*-Di-quinolin-6-yl-9H-purine-2,6-diamine;
N*6*-Benzhydryl-N*2*-benzothiazol-6-yl-9H-purine-2,6-diamine;
N*6*-Phenyl-N*2*-quinolin-6-yl-9H-purine-2,6diamine;
N*6*-Benzhydryl-N*2*-quinolin-6-yl-9H-purine-2,6-diamine;
N*6*-tert-Butyl-N*2*-naphthalen-2-yl-9H-purine-2,6-diamine;
N*6*-tert-Butyl-N*2*-(2-methyl-quinolin-6-yl)-9H-purine-2,6-diamine;
N*6*-tert-Butyl-N*2*-(2-methyl-benzothiazol-5-yl)-9H-purine-2,6-diamine;
N*6*-tert-Butyl-N*2*-(2-tert-butyl-benzothiazol-6-yl)-9H-purine-2,6-diamine;
N*6*-tert-Butyl-N*2*-(2-methyl-benzothiazol-6-yl)-9H-purine-2,6-diamine;
N*2*-(4-Benzothiazol-2-yl-phenyl)-N*6*-tert-butyl-9H-purine-2,6-diamine;
N*6*-tert-Butyl-N*2*-(6-methoxy-naphthalen-2-yl)-9H-purine-2,6-diamine;
N*6*-tert-Butyl-N*2*-[6-(2-morpholin-4-yl-ethoxy)-naphthalen-2-yl]-9H-purine-2,6-diamine;
N*6*-tert-Butyl-N*2*-(2-methyl-1H-indol-5-yl)-9H-purine-2,6-diamine;
N*6*-tert-Butyl-N*2*-(1H-indol-5-yl)-9H-purine-2,6-diamine;
N*2*-Benzothiazol-6-yl-N*6*-tert-butyl-8-methyl-9H-purine-2,6-diamine;
N*2*-Benzothiazol-6-yl-N*6*-cycloheptyl-8-methyl-9H-purine-2,6-diamine;
N*2*-Benzothiazol-6-yl-N*6*-tert-butyl-8-ethyl-9H-purine-2,6-diamine;
N*2*-Benzothiazol-6-yl-8-ethyl-N*6*-(2,2,2-trifluoro-1-methyl-ethyl)-9H-purine-2,6-diamine;
Benzothiazol-6-yl-[8-ethyl-6-(2-methyl-pyrrolidin-1-yl)-9H-purin-2-yl]-amine;
3-[2-(Benzothiazol-6-ylamino)-8-ethyl-9H-purin-6-ylamino]-butyramide;
3-[2-(Benzothiazol-6-ylamino)-8-ethyl-9H-purin-6-ylamino]-butyric acid ethyl ester;
N*2*-Benzothiazol-6-yl-N*6*-tert-butyl-8-propyl-9H-purine-2,6-diamine;
N*2*-Benzothiazol-6-yl-N*6*-tert-butyl-8-isopropyl-9H-purine-2,6-diamine;
N*2*-Benzothiazol-6-yl-N*6*-tert-butyl-8-cyclopentyl-9H-purine-2,6-diamine;
6-(6-tert-Butylamino-8-ethyl-9H-purin-2-ylamino)-chromen-2-one;
N*6*-tert-Butyl-8-ethyl-N*2*-(2-methylsulfanyl-benzothiazol-6-yl)-9H-purine-2,6-diamine;
N*6*-tert-Butyl-8-ethyl-N*2*-[2-(2-morpholin-4-yl-ethoxy)-benzothiazol-6-yl]-9H-purine-2,6-diamine;
N*6*-tert-Butyl-8-isopropyl-N*2*-{1-methylene-3-[5-methyl-2-(2-morpholin-4-yl-ethoxy)-thiazol-4-yl]-allyl}-9H-purine-2,6-diamine;
N*6*-tert-Butyl-8-cyclopropyl-N*2*-{1-methylene-3-[5-methyl-2-(2-morpholin-4-yl-ethoxy)-thiazol-4-yl]-allyl}-9H-purine-2,6-diamine;
N*6*-tert-Butyl-8-cyclopentyl-N *2*-{1-methylene-3-[5-methyl-2-(2-morpholin-4-yl-ethoxy)-thiazol-4-yl]-allyl}-9H-purine-2,6-diamine;
N*2*-Benzothiazol-6-yl-6-yl-N*6*-tert-butyl-8-cyclopropyl-9H-purine-2,6-diamine;
N*2*-Benzothiazol-6-yl-N*6*-tert-butyl-9H-purine-2,6,8-triamine;
N*6*-tert-Butyl-N*2*-naphthalen-2-yl-9H-purine-2,6,8-triamine;
N*6*-tert-Butyl-N*8*-methyl-N*2*-naphthalen-2-yl-9H-purine-2,6,8-triamine;
1-[2-(Benzothiazol-6-ylamino)-6-tert-butylamino-9H-purin-8-yl]-ethanone;
N*6*-tert-Butyl-N*2*-quinolin-6-yl-9H-purine-2,6-diamine;
N*2*-Benzo[1,2,5]thiadiazol-5-yl-N*6*-tert-butyl-9H-purine-2,6-diamine;
N*6*-tert-Butyl-N*2*-(2-methyl-benzothiazol-6-yl)-9H-purine-2,6-diamine;
N*2*-Benzo[1,2,5]thiadiazol-5-y-N*6*-cycloheptyl-9H-purine-2,6-diamine;
N*6*-Cycloheptyl-N*2*-(2-methyl-benzothiazol-6-yl)-9H-purine-2,6-diamine;
N*2*-Benzothiazol-6-yl-N*6*-([1,1-dimethyl-propyl)-9H-purine-2,6-diamine;
N*6*-(1,1-Dimethyl-propyl)-N*2*-quinolin-6-yl-9H-purine-2,6-diamine;
N*2*-Benzo[1,2,5]thiadiazol-5-y-N*6*-(1,1-dimethyl-propyl)-9H-purine-2,6-diamine;
N*6*-(1,1-Dimethyl-propyl)-N*2*-(2-methyl-benzothiazol-6-yl)-9H-purine-2,6-diamine;
N*2*-Benzothiazol-6-yl-N*6*-[2-(4-fluoro-phenyl)-1,1-dimethyl-ethyl]-9H-purine-2,6-diamine;
N*6*-[2-(4-Fluoro-phenyl)-1,1-dimethyl-ethyl]-N*2*-quinolin-6-yl-9H-purine-2,6-diamine;
N*2*-Benzo[1,2,5]thiadiazol-5-yl-N*6*-[2-(4-fluoro-phenyl)-1,1-dimethyl-ethyl]-9H-purine-2,6-diamine;
N*6*-[2-(4-Fluoro-phenyl)-1,1-dimethyl-ethyl]-N*2*-(2-methyl-benzothiazol-6-yl)-9H-purine-2,6-diamine;
Benzothiazol-6-yl-[6-(4-pyridin-3-yl-piperazin-1-yl)-9H-purin-2-yl]-amine;
Benzothiazol-6-yl-[6-(4-pyridin-2-yl-piperazin-1-yl)-9H-purin-2-yl]-amine;
[6-(4-Pyridin-2-yl-piperazin-1-yl)-9H-purin-2-yl]-quinolin-6-yl-amine;
Benzo[1,2,5]thiadiazol-5-yl-[6-(4-pyridin-2-yl-piperazin-1-yl)-9H-purin-2-yl]-amine;
(2-Methyl-benzothiazol-6-yl)-[6-(4-pyridin-2-yl-piperazin-1-yl)-9H-purin-2-yl]-amine;
Quinolin-6-yl-[6-(2,3,5,6-tetrahydro-[1,2′]bipyrazinyl-4-yl)-9H-purin-2-yl]-amine;
N*2*-Benzothiazol-6-yl-N*6*-tert-butyl-9H-purine-2,6-diamine;
N*2*-Benzothiazol-6-yl-N*6*-cycloheptyl-9H-purine-2,6-diamine;
N*6*-Cycloheptyl-N*2*-quinolin-6-yl-9H-purine-2,6-diamine;
2-[2-(Benzothiazol-6-ylamino)-9H-purin-6-ylamino]-2-methyl-propan-1-ol;
8-Bromo-N*6*-tert-butyl-N*2*-naphthalen-2-yl-9H-purine-2,6-diamine;
N*6*-tert-Butyl-N*2*-naphthalen-2-yl-8-vinyl-9H-purine-2,6-diamine;
8-Allyl-N*6*-tert-butyl-N*2*-naphthalen-2-yl-9H-purine-2,6-diamine;
N*6*-tert-Butyl-8-methyl-N*2*-naphthalen-2-yl-9H-purine-2,6-diamine;
N*6*-tert-Butyl-8-ethyl-N*2*-naphthalen-2-yl-9H-purine-2,6-diamine;
N*6*-tert-Butyl-8-isobutyl-N*2*-naphthalen-2-yl-9H-purine-2,6-diamine;
and pharmaceutically acceptable salts thereof.

16. A compound selected from the group consisting of:
(2-Chloro-9H-purin-6-yl)-cycloheptyl-amine;
2-(2-Chloro-9H-purin-6-ylamino)-2-methyl-propan-1-ol;
Adamantan-2-yl-(2-chloro-9H-purin-6-yl)-amine;
(2-Chloro-9H-purin-6-yl)-cyclooctyl-amine;
4-(2-Chloro-9H-purin-6-ylamino)-cyclohexanol;
(2-Chloro-9H-purin-6-yl)-quinolin-6-yl-amine;
Benzhydryl-(2-chloro-9H-purin-6-yl)-amine;
N*6*-Benzhydryl-N*2*-quinolin-6-yl-9-(tetrahydro-pyran-2-yl)-9H-purine-2,6-diamine;
N*2*-Benzothiazol-6-yl-N*6*-tert-butyl-9-(tetrahydro-pyran-2-yl)-9H-purine-2,6-diamine;
N*6*-tert-Butyl-N*2*-naphthalen-2-yl-9-(tetrahydro-pyran-2-yl)-9H-purine-2,6-diamine;
N*6*-tert-Butyl-N*2*-(2-methyl-quinolin-6-yl)-9-(tetrahydro-pyran-2-yl)-9H-purine-2,6-diamine;
N*6*-tert-Butyl-N*2*-(2-methyl-benzothiazol-5-yl)-9-(tetrahydro-pyran-2-yl)-9H-purine-2,6-diamine;
N*6*-tert-Butyl-N*2*-(2-tert-butyl-benzothiazol-6-yl)-9-(tetrahydro-pyran-2-yl)-9H-purine-2,6-diamine;
N*6*-Cycloheptyl-N*2*-quinolin-6-yl-9-(tetrahydro-pyran-2-yl)-9H-purine-2,6-diamine;
N*2*-(4-Benzothiazol-2-yl-phenyl)-N*6*-tert-butyl-9-(tetrahydro-pyran-2-yl)-9H-purine-2,6- diamine;
N*6*-tert-Butyl-N*2*-(6-methoxy-naphthalen-2-yl)-9-(tetrahydro-pyran-2-yl)-9H-purine-2,6-diamine;
N*6*-tert-Butyl-N*2*-[6-(2-morpholin-4-yl-ethoxy)-naphthalen-2-yl]-9-(tetrahydro-pyran-2-yl)-9H-purine-2,6-diamine;
5-{9-[Bis-(4-methoxy-phenyl)-methyl]-6-tert-butylamino-9H-purin-2-ylamino)-2-methyl-indole-1-carboxylic acid tert-butyl ester;
9-[Bis-(4-methoxy-phenyl)-methyl]-N*6*-tert-butyl-N*2*-(l H-indol-5-yl)-9H-purine-2,6 diamine;
9-[Bis-(4-methoxy-phenyl)-methyl]-N*6*-tert-butyl-8-ethyl-N*2*-[2-(2-morpholin-4-yl-ethoxy)-benzothiazol-6-yl]-9H-purine-2,6-diamine;
N*6*-tert-Butyl-8-ethyl-N*2*-[2-(2-morpholin-4-yl-ethoxy)-benzothiazol-6-yl]-9-(tetrahydro-pyran-2-yl)-9H-purine-2,6-diamine;
N*6*-tert-Butyl-8-isopropyl-N*2*-{1-methylene-3-[5-methyl-2-(2-morpholin-4-yl-ethoxy)-thiazol-4-yl]-allyl}-9-(tetrahydro-pyran-2-yl)-9H-purine-2,6-diamine;
N*6*-tert-Butyl-8-cyclopropyl-N*2*-{1-methylene-3-[5-methyl-2-(2-morpholin-4-yl-ethoxy)-thiazol-4-yl]-allyl}-9-(tetrahydro-pyran-2-yl)-9H-purine-2,6-diamine;
N*6*-tert-Butyl-8-cyclopentyl-N*2*-{1-methylene-3-[5-methyl-2-(2-morpholin-4-yl-ethoxy)-thiazol-4-yl]-allyl}-9-(tetrahydro-pyran-2-yl)-9H-purine-2,6-diamine;
N*2*-Benzothiazol-6-yl-N*6*-tert-butyl-8-cyclopropyl-9-(tetrahydro-pyran-2-yl)-9H-purine-2,6-diamine;
N*2*-Benzothiazol-6-yl-N*6*-tert-butyl-N*B*-(2,4-dimethoxy-benzyl)-9-(tetrahydro-pyran-2-yl)-9H-purine-2,6,8-triamine;
N*6*-tert-Butyl-N*8*-(2,4-dimethoxy-benzyl)-N*2*-naphthalen-2-yl-9-(tetrahydro-pyran-2-yl)-9H-purine-2,6,8-triamine;
N*6*-tert-Butyl-N *8*-(2,4-dimethoxy-benzyl)-N*8*-methyl-N*2*-naphthalen-2-yl-9-(tetrahydro-pyran-2-yl)-9H-purine-2,6,8-triamine;
N*2*-Benzothiazol-6-yl-N*6*-tert-butyl-8-(1-ethoxy-vinyl)-9-(tetrabydro-pyran-2-yl)-9H-purine-2,6-diamine;
tert-Butyl-(2-chloro-8-methyl-9H-purin-6-yl)-amine;
(2-Chloro-8-methyl-9H-purin-6-yl)-cycloheptyl-amine;
tert-Butyl-(2-chloro-8-ethyl-9H-purin-6-yl)-amine;
(2-Chloro-8-ethyl-9H-purin-6-yl)-(2,2,2-trifluoro-1-methyl-ethyl)-amine;
2-Chloro-8-ethyl-6-(2-methyl-pyrrolidin-1-yl)-9H-purine;
3-(2-Chloro-8-ethyl-9H-purin-6-ylamino)-butyramide;
3-(2-Chloro-8-ethyl-9H-purin-6-ylamino)-butyric acid ethyl ester;
tert-Butyl-(2-chloro-8-propyl-9H-purin-6-yl)-amine;
tert-Butyl-(2-chloro-8-isopropyl-9H-purin-6-yl)-amine;
tert-Butyl-(2-chloro-8-cyclopentyl-9H-purin-6-yl)-amine;
tert-Butyl-(2-chloro-8-cyclopropyl-9H-purin-6-yl)-amine;
Benzhydryl-[2-chloro-9-(tetrahydro-pyran-2-yl)-9H-purin-6-yl]-amine;
tert-Butyl-[2-chloro-9-(tetrahydro-pyran-2-yl)-9H-purin-6-yl]-amine;
[2-Chloro-9-(tetrahydro-pyran-2-yl)-9H-purin-6-yl]-cycloheptyl-amine;
{9-[Bis-(4-methoxy-phenyl)-methyl]-2-chloro-9H-purin-6-yl)-tert-butyl-amine;
{9-[Bis-(4-methoxy-phenyl)-methyl]-2-chloro-8-ethyl-9H-purin-6-yl)-tert-butyl-amine;
tert-Butyl-[2-chloro-8-ethyl-9-(tetrahydro-pyran-2-yl)-9H-purin-6-yl]-amine;
tert-Butyl-[2-chloro-8-isopropyl-9-(tetrahydro-pyran-2-yl)-9H-purin-6-yl]-amine;
tert-Butyl-[2-chloro-8-cyclopropyl-9-(tetrahydro-pyran-2-yl)-9H-purin-6-yl]-amine;
tert-Butyl-[2-chloro-8-cyclopentyl-9-(tetrahydro-pyran-2-yl)-9H-purin-6-yl]-amine;
tert-Butyl-[2-chloro-8-cyclopropyl-9-(tetrahydro-pyran-2-yl)-9H-purin-6-yl]-amine;
N*6*-tert-Butyl-2-chloro-N*8*-(2,4-dimethoxy-benzyl)-9-(tetrahydro-pyran-2-yl)-9H-purine-6,8-diamine;
N*6*-tert-Butyl-2-chloro-N*8*-(2,4-dimethoxy-benzyl)-N*8*-methyl-9-(tetrahydro-pyran-2-yl)-9H-purine-6,8-diamine;
tert-Butyl-[2-chloro-8-(1-ethoxy-vinyl)-9-(tetrahydro-pyran-2-yl)-9H-purin-6-yl]-amine;
9-[Bis-(4-methoxy-phenyl)-methyl]-2,6-dichloro-9H-purine;
N-(4-Amino-2,6-dichloro-pyrimidin-5-yl)-acetimidic acid ethyl ester;
N-(4-Amino-2,6-dichloro-pyrimidin-5-yl)-propionimidic acid ethyl ester;
N-(4-Amino-2,6-dichloro-pyrimidin-5-yl)-butyrimidic acid methyl ester;
N-(4-Amino-2,6-dichloro-pyrimidin-5-yl)-2-methyl-propionimidic acid ethyl ester;
N-(4-Amino-2,6-dichloro-pyrimidin-5-yl)-cyclopentanecarboximidic acid ethyl ester;
N-(4-Amino-2,6-dichloro-pyrimidin-5-yl)-cyclopropanecarboximidic acid ethyl ester;
6-(2-Morpholin-4-yl-ethoxy)-naphthalen-2-ylamine;
4-[2-(6-Bromo-naphthalen-2-yloxy)-ethyl]-morpholine;
5-Amino-2-methyl-indole-1-carboxylic acid tert-butyl ester;
2-Methyl-5-nitro-indole-1-carboxylic acid tert-butyl ester;
2-(2-Morpholin-4-yl-ethoxy)-benzothiazol-6-ylamine;
2-(2-Morpholin-4-yl-ethoxy)-6-nitro-benzothiazole;
[8-Bromo-2-chloro-9-(tetrahydro-pyran-2-yl)-9H-purin-6-yl]-tert-butyl-amine;
8-Bromo-2,6-dichloro-9-(tetrahydro-pyran-2-yl)-9H-purine; and
2,6-Dichloro-8-(1-ethoxy-vinyl)-9-(tetrahydro-pyran-2-yl)-9H-purine.

17. (canceled)

18. A process to prepare a compound according to claim 4 comprising:
(A) reacting a substituted 9H-purin-6-ylamine of Formula (II) with an hereroaryl/aryl-amine to form a compound of Formula (I), or; 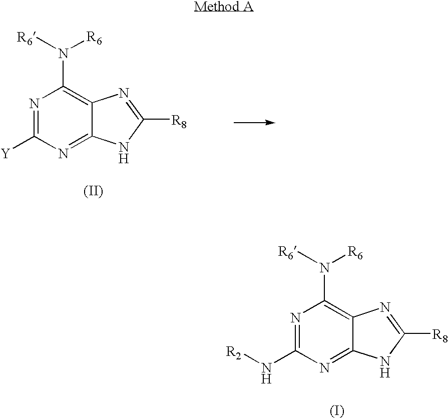
(B) reacting a substituted 9H-purin-6-yl of Formula (IV), substituted with a protecting group, R₉, with an hereroaryl/aryl-amine using preferably a palladium catalysed S_{N}Ar reaction and removing of the protecting group to form a compound of Formula (I); or 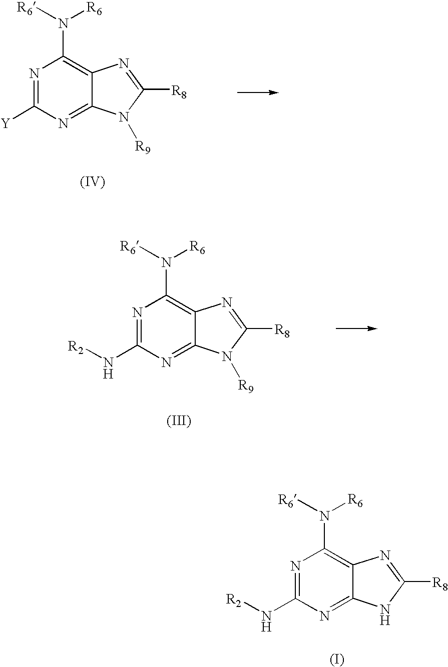
(C) reacting, in a solid phase, using a Rink acid resin, a substituted 9H-purin-6-yl, with an appropriate amine to afford substitution at the 6 position, followed by reaction with an hereroaryl/aryl-amine using preferably a palladium catalysed S_{N}Ar reaction to afford substitution at the 2 position, cleavage from the resin and purification: 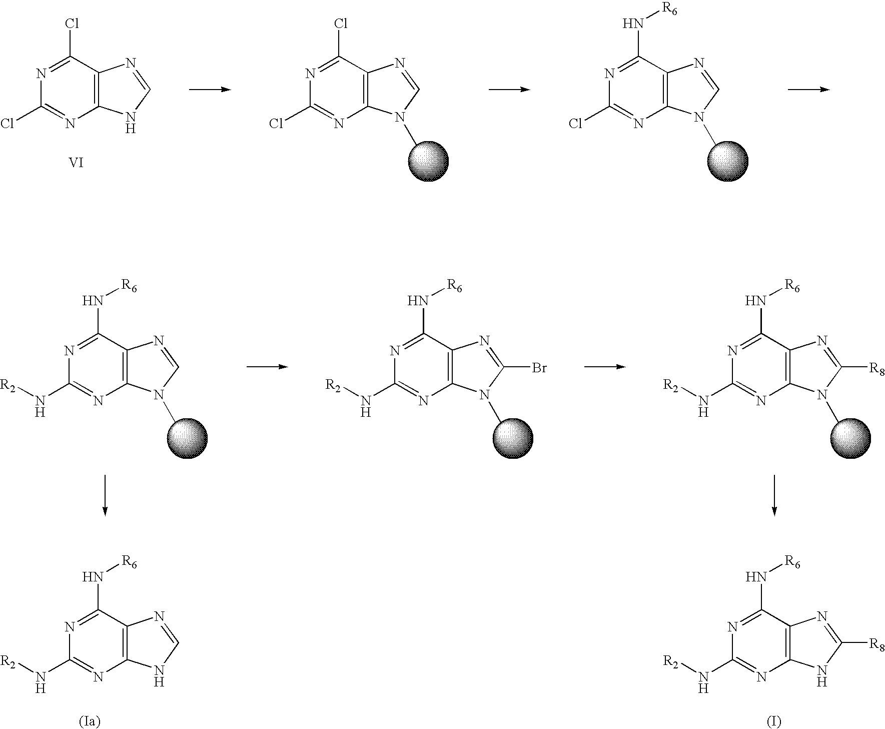
and, if desired, after reaction (A), (B) or (C), transforming an obtainable compound of Formula (I) into a different compound of Formula (I); transforming a salt of an obtainable compound of Formula (I) into the free compound or a different salt or an obtainable free compound of Formula (I) into a salt; and/or separating an obtainable mixture of isomers of compounds of Formula (I) into the individual isomers,
wherein R′₆, R₆, R₂, and R₈ are as defined in claim 4; and Y and R₉ are protecting groups.
